(19) United States
(12) Patent Application Publication
(10) Pub. No.: US 2026/0015623 A1
(43) Pub. Date: Jan. 15, 2026

(54) NUCLEIC ACID CONSTRUCTS FOR GENOME EDITING

(71) Applicant: The State of Israel, Ministry of Agriculture & Food Security, Agricultural Research Organization (AR, Rishon-LeZion (IL)

(72) Inventors: Gilor KELLY, Beit-Elazari (IL); Elena PLESSER, Meitar (IL); Eyal BDOLACH, Rehovot (IL); Amir SHERMAN, Beit Zayit (IL); Nir CARMI, Rehovot (IL); Yoram EYAL, Mesilat Zion (IL)

(73) Assignee: The State of Israel, Ministry of Agriculture & Food Security, Agricultural Research Organization (AR, Rishon-LeZion (IL)

(21) Appl. No.: 19/263,645

(22) Filed: Jul. 9, 2025

Related U.S. Application Data
(60) Provisional application No. 63/668,814, filed on Jul. 9, 2024.
(60) Provisional application No. 63/786,353, filed on Apr. 10, 2025.

(51) Int. Cl. C12N 15/82; C12N 9/22; C12N 15/11

(52) U.S. Cl. CPC C12N 15/8205; C12N 9/226; C12N 15/111; C12N 15/8262; C12N 2310/20

(57) ABSTRACT

A nucleic acid construct or construct system for genome editing is provided. Also provided are non-transgenic plants having been edited accordingly and methods of producing such plants.

### RELATED APPLICATIONS

This application claims the benefit of priority under 35 USC § 119(e) of U.S. Provisional Patent Application Nos. 63/786,353 filed on Apr. 10, 2025 and 63/668,814 filed on Jul. 9, 2024, the contents of which are all incorporated by reference as if fully set forth herein in their entirety.

### SEQUENCE LISTING STATEMENT

The XML file, entitled 104212SequenceListing.xml, created on Jul. 8, 2025, comprising 88,650 bytes, submitted concurrently with the filing of this application is incorporated herein by reference.

### FIELD AND BACKGROUND OF THE INVENTION

The present invention, in some embodiments thereof, relates to nucleic acid constructs for genome editing.

Conventional cross-breeding has long been the predominant method for developing new elite varieties in fruit trees. Yet, this approach is challenging and limited due to the long juvenile phase, high levels of genetic heterogeneity, and the need to mount multiple traits. In the case of Citrus, the time required to establish a new variety could span up to a decade, delaying the overall cultivation progress (Song et al., 2019, Alvarez et al., 2021). Furthermore, it limits the ability to rapidly introduce specific traits in response to emerging needs, such as contesting disease outbreaks. Consequently, targeted breeding approaches are essential for efficient and accelerated cultivar production in Citrus.

Clustered Regulatory Interspaced Short Palindromic Repeats (CRISPR)/Cas9-genome editing technology offers a promising opportunity for precise trait modification and an alternative to the traditional breeding program (Zhou et al., 2020, Alvarez et al., 2021). However, its integration into fruit tree species remains an ongoing effort. Currently, the process requires callus formation followed by regeneration through tissue culture (Dutt et al., 2022, Huang et al., 2022a), which is time-consuming, laborious, and cost-ineffective. Furthermore, the nature of tissue culturing results in non-commercial transgenic plants, thus limiting its products only to research.

To overcome these challenges, the in-planta strategy was recently introduced (Zhang et al., 2017b, Rizwan et al., 2021, Lian et al., 2022, Hao et al., 2024). Using this method, a Genome Editing (GE) cassette and a Developmental Regulators (DRs) cassette are co-delivered to Cas9-expressing plants (Maher et al., 2020). The expression of DRs, such as WUS, STM, Ipt, and PLT5, was shown to induce developmental changes in somatic cells, leading to the acquisition of meristematic identity (Zhang et al., 2017a, Maher et al., 2020, Eshed Williams, 2021, Nasti and Voytas, 2021, Lian et al., 2022). In tobacco, the combined effects of GE and DRs facilitates de novo edited meristem induction, which can later develop into a fully mutated shoot (Maher et al., 2020). Yet, this strategy was so far not tested in fruit trees, and was shown to be successful when using transgenic plants pre-expressing Cas9 (Maher et al., 2020).

In addition to genome editing, obtaining transgene-free plants is crucial for the development of new cultivars. To achieve transgene-free plants, passive and active approaches can be employed. In the passive approach, in which the selection step is avoided, the mutation occurs via transient activation of the genome-editing elements. The second approach, however, is aimed at actively impairing the T-DNA integration process into the genome.

WO/2021/026081 discloses genome editing in Citrus using CRISPR-ribonucleoprotein complexes where transfection is done on isolated protoplasts.

WO/2020/006112 discloses induction of meristem by introducing developmental regulators to perform in planta genome editing in transgenic plants that constitutively express transgenic Cas9 (T₀).

### SUMMARY OF THE INVENTION

According to an aspect of some embodiments of the present invention there is provided a method of producing a genomically edited plant or part thereof comprising contacting the plant or part thereof with the nucleic acid construct or construct system under conditions which allow editing and regeneration, wherein the plant is non-transgenic at T₀, wherein the nucleic acid construct or construct system comprises:
(i) at least one nucleic acid sequence encoding a genome editing agent for introducing a sequence variation in a target gene of interest in a plant cell; and
(ii) at least one nucleic acid sequence encoding at least one gene being a developmental regulator.

According to some embodiments of the invention, a progeny of the genomically edited plant is non-transgenic.

According to some embodiments of the invention, the nucleic acid construct or construct system of claim 1, further comprising (iii) at least one nucleic acid sequence encoding at least one gene for facilitating Agrobacterium delivery into nuclei of the plant cells.

According to an aspect of some embodiments of the present invention there is provided a nucleic acid construct or construct system comprising:
(i) at least one nucleic acid sequence encoding a genome editing agent for introducing a sequence variation in a target of interest in a plant cell; and
(ii) at least one nucleic acid sequence encoding at least one gene being a developmental regulator;

According to some embodiments of the invention, the nucleic acid construct or construct system further comprises (iii) at least one nucleic acid sequence encoding at least one gene for facilitating Agrobacterium delivery into nuclei of the plant cells.

According to some embodiments of the invention, the nucleic acid construct or construct system, further comprises (iv) at least one nucleic acid sequence for preventing T-DNA integration.

According to some embodiments of the invention, the at least one nucleic acid sequence for preventing T-DNA integration is directed against Polymerase θ (Polθ).

According to some embodiments of the invention, the at least one nucleic acid sequence for preventing T-DNA integration is an integral part of the construct or system.

According to some embodiments of the invention, the at least one nucleic acid sequence for preventing T-DNA integration is auxiliary to the construct or system.

According to some embodiments of the invention, the at least one nucleic acid sequence for preventing T-DNA integration is an RNA silencing agent or a genome editing agent.

According to some embodiments of the invention, the genome editing agent is of the CRISPR-Cas system.

According to some embodiments of the invention, the Cas is selected from the group consisting of Cas9 and Cas12a, Cas12b, Cas13 and CasX.

According to some embodiments of the invention, the genome editing agent comprises gRNA.

According to some embodiments of the invention, the gene for facilitating Agrobacterium delivery into nuclei of the plant cells is a bacterial gene.

According to some embodiments of the invention, the gene for facilitating Agrobacterium delivery into nuclei of the plant cells is a bacterial gene.

According to some embodiments of the invention, the nucleic acid construct or construct system comprises at least one copy (e.g., 2, 3, 4, 5) of (i), (ii), (iii) or (iv).

According to some embodiments of the invention, the (i) is present in at least two constructs of the construct system.

According to some embodiments of the invention, the nucleic acid construct or construct system comprises cis acting regulatory elements operatively linked to at least one nucleic acid sequences as described herein.

According to some embodiments of the invention, at least 2 of the cis acting regulatory element are different.

According to some embodiments of the invention, the cis acting regulatory elements are promoters.

According to some embodiments of the invention, the cis acting regulatory elements or promoters which are operably linked to (i) are active in cells undergoing cell division (e.g., Yao).

According to an aspect of some embodiments of the present invention there is provided a plant or part thereof comprising the nucleic acid construct or construct system as described herein.

According to some embodiments of the invention, the plant or plant part of claim is non-transgenic.

According to some embodiments of the invention, the plant or part thereof is homozygous, bi-allelic or multi-allelic for the sequence variation.

According to some embodiments of the invention, the plant is an annual plant.

According to some embodiments of the invention, the plant is a perennial plant.

According to some embodiments of the invention, the plant is a tree.

According to some embodiments of the invention, the plant is a monocot tree.

According to some embodiments of the invention, the plant is a fruit tree.

According to some embodiments of the invention, the plant is a Citrus.

According to some embodiments of the invention, the plant is Cannabis.

According to an aspect of some embodiments of the present invention there is provided a cell comprising the nucleic acid construct or construct system as described herein.

According to some embodiments of the invention, the cell is an Agrobacterium.

According to an aspect of some embodiments of the present invention there is provided a method of producing a genomically edited plant or part thereof comprising contacting the plant or part thereof with the nucleic acid construct or construct system as described herein under conditions which allow editing and regeneration.

According to some embodiments of the invention, the conditions comprise exposing to blue light prior to the contacting.

According to some embodiments of the invention, the conditions comprise maintaining the plant or part thereof in the dark to promote regeneration following the contacting and prior to exposing to light.

According to some embodiments of the invention, the contacting is at an apical site of a young seedling.

According to an aspect of some embodiments of the present invention there is provided a method of producing a plant, the method comprising grafting the plant part as described herein.

According to an aspect of some embodiments of the present invention there is provided a method of producing a plant, the method comprising regenerating the plant part as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F shows the CRISPRUS genetic system. (A-C) Plasmids 501 and 502. These plasmids incorporate Cas9 under global promoter, a genome editing booster cassette (B, pU6-gRNA-scaffold-terminator unit followed by two tRNA-based multiplexed editing units expressed under the endogenous U6 promoter), selection markers (antibiotic resistance, GFP) and a (C) cassette containing developmental regulators (501; WUS and STM, 502; ipt). (D) Plasmid 216 includes factors for enhanced de novo meristem induction (LAS, RAX1, EXB1, ROX, and ARR1). (E) Plasmid 409 contains Cas9 under the YAO promoter, along with VIP1, and VirE2 for improved Agrobacterium delivery. (F) Summary of the CRISPRUS genetic system, highlighting key transcriptional units employed in this study.

FIGS. 2A-D show gene expression analysis and confocal imaging at three-time points following agro infiltration of plasmids 502 and 216 into Carrizo WT leaves. (A) Transcriptional units are illustrated for each plasmid. Representative genes tested for their expression are colored-coded (blue-502 and shown in 2B, pink-216 and shown in 2C). (B) Expression analysis of Cas9, GFP, and IPT at 3 days, 6 days and 10 days following agro-infiltration. (C) Expression analysis of LAS, EXB1, and ARR1 at 3 days, 6 days and 10 days following agro-infiltration. (B-C) Data points are means±SE (n=6). Different letters indicate significant differences (Tukey's HSD test, P<0.05). (D) Distribution of GFP on day ten following agro-infiltration. Panels are GFP fluorescence (stained green, top left), white-light (top right), chlorophyll-autofluorescence (stained magenta, bottom left) and merged (bottom right). Bar=50 μm.

FIGS. 3A-G shows an overview of the method of genome editing according to some embodiments of the invention and regeneration efficiency of the CRISPRUS system. (A) A method overview: The regeneration process involves cutting the stem of four-month-old seedlings below the first true leaf (2), applying a mixture of agrobacteria to the cut site for 1 hour (3), and incubating the seedlings in the dark for ten days (to promote regeneration) before transferring them to the light (4). Shoot emergence begins 30-60 days post-transformation. (B-D) Regeneration efficiency of plants treated with agro lacking developmental regulators (B), or the CRISPRUS system, evaluated 60 days' post transformation (C). Each column represents an independent plant, and filled squares denote the number of shoots per plant, emerging either from the top (orange) or the side (green). An illustration of seedlings with top shoots (orange square) and side shoots (green squares) is provided in (D). (E) Regeneration efficiency comparison. Evaluation of plants with no treatment (shoots removed), plants treated with agrobacteria (without developmental regulators) or CRISPRUS-treated plants. The rates of plants with no regeneration, side only shoots, top shoots and plants containing two shoots or more emerging from the top, are presented. (F-G) Representative images of top shoot regeneration. (F) Representative images of regenerated plants displaying none- to multiple shoots. (G) Over-regeneration resulting in excessive shoot production and callus formation.

FIGS. 4A-D shows genetic transformation and gene-editing using the in-planta system. (A) Induction of transgenic shoots expressing GFP. Panels display merged images of GFP (stained green) and chlorophyll-autofluorescence (stained magenta). The bar scale is indicated for each image. (B) Regenerated shoots presenting albino or a sectorial chimeric morphology containing adjacent WT and albino axils. Sample 17-1 shows both albino and chimeric leaves. Orange arrows denote albino stripes extended from the base to the shoot tip. (C) DNA sequences of gene edits in regenerated shoots of 17-1, 17-2 (SEQ ID Nos: 58-62). gRNA's (blue), PAM site (green), and primers flanking the expected mutated site (orange) displayed above. Deletions and insertion (pink) for exon 14 of CsPDS are indicated. (D) Experiment summary. Evaluation of CRISPRUS-treated plants. The rates of plants with no regeneration, side only shoots, top shoots, transgenic and albino plants are presented.

FIGS. 5A-D show phenotypic responses of regenerated shoots to different treatments. (A) Representative images of plants displaying a complete albino or a partial albino (chimeric) phenotypes in response to various treatments, including normal, half optical density (½OD), without a selection step (no selection), or with exposure to blue light prior to transformation. (B) Experiment summary. Evaluation of CRISPRUS-treated plants in response to different treatments. The rates of plants with no regeneration, side only shoots, top shoots, and the frequency of albino plants regenerated in response to different treatments are presented. (C) Representative CsPDS sequences displaying gene edits in specific exons in regenerated shoots (samples 36-1, 36-2 and BL5). Sample BL5 demonstrates multiallelic mutations in four different exons. The gRNA's sequences (bold), PAM site (green), Deletions (pink) and insertions (blue) for exons #3-#5, and #14 of CsPDS are indicated (SEQ ID NOS. 63-76) (D) Representative images of WT and albino plant following tissue lysis. The gRNA sequences are in bold.

FIGS. 6A-D show the in-planta system in Duncan. (A) Induction of transgenic shoots expressing GFP. Panels are merged images of GFP (stained green) and chlorophyll-autofluorescence (stained magenta). Bar scales are indicated for each image. (B) Transgenic plant displaying a sectorial chimeric morphology divided by the central leaf vein. GFP fluorescence is observed in the white-albino section of the leaf. (C) Transgenic plant containing partial editing with a milky-yellowish phenotype and a mix of WT and GFP-expressing cells. (B-C) Panels are GFP fluorescence, white light, chlorophyll-autofluorescence, and merged. Scale bars are specified in each image. (D) Experiment summary. Evaluation of none-treated and CRISPRUS-treated Duncan plants. The rates of plants with no regeneration, side only shoots, top shoots, and plants containing two shoots or more emerging from the top, are presented.

FIGS. 7A-D show an active approach for obtaining transgene-free plants. (A) Phylogenetic tree of DNA POLYMERASE (POL) genes from Citrus and Arabidopsis plants. Different Pol genes from Arabidopsis are listed in Table 1. AtPOLθ and its Citrus homolog, Cs4g19990, are highlighted in orange. (B) Reconstruction of the CRISPRUS system to include a segment of RNAi-Polθ in three of the four vectors applied. (C) Representative images of albino plants with a CsPDS-impaired gene were treated with the reconstructed system, as detailed in (B). (D) Summary of Taq-Man analysis using probes specific for GFP (FAM, indicates transgenic plants) and CsACTIN (CY5, indicates DNA quality). Albino (mutated) samples from several experiments were tested (FIGS. 3A-G to 4A-D). ‘No selection’ refers to the passive method (FIG. 5A-D), and ‘Polθ1.1’ and ‘Polθ1.2’ refer to the active RNAi-Polθ (C) method. All samples from no selection and RNAi-Polθ treatments are transgene-free (negative for the presence of GFP, D). Selected samples from other experiments are presented as well. Sample 17-2 is transgene-free.

FIGS. 8A-B show genetic transformation and gene editing of Carrizo plants using vacuum. (A) Representative images of shoot regeneration from infected sites. Images were taken five weeks after the transformation. (B) Regenerated shoots display a complete albino (mutated) phenotype and GFP fluorescence. Panels are white light, GFP fluorescence (stained green), and RFP fluorescence (stained red) to rule out non-specific autofluorescence. Bar=1 mm.

FIGS. 9A-C show the identification of transgene-free, genome-edited plants. (A) Genotyping workflow. Following DNA extraction, high-resolution melting (HRM) analysis was used to identify candidates potentially harboring gene editing events. Candidates were then sent for sequencing for mutation verification, followed by PCR and Taq-Man analysis for testing transgene presence. Samples #23 and #81 represent plants displaying CsPDS gene editing events, that are either transgenic or transgene-free, respectively. (B) CsPDS sequences of samples #23 and #81, displaying gene editing events in exon3 and exon13 of regenerated shoots. The gRNA sequences (bold), PAM site (green), deletions (pink) and insertions (blue) are indicated. (C) PCR analysis of samples #23 and #81 for the detection of SpCas9. CsACTIN served as a reference gene. NTC; no template control. SEQ ID Nos: 78-92 are indicated in the figure. Primers are listed in the Table 3, below.

FIGS. 10A-C show the effect of developmental regulators on the regeneration and transformation efficiencies of different cultivars. (A) Schematic description of the vectors assembled for this assay. A RUBY unit, together with NptII transcriptional unit, were assembled together with four different developmental regulators (DRs; IPT, WUS-STM, GRF4-GIF1 and PLT5). (B) Representative images of betalain-accumulating plants following transformation. Plants were either chimeric or completely transformed. (C) Regeneration and transformation efficiency of Carrizo, Duncan, Foster and Hudson cultivars following transformation with RUBY-IPT, RUBY-WUS-STM, RUBY-GRF4-GIF1 and RUBY-PLT5. Gray columns represent regeneration efficiency and pink columns (inset) represents transformation efficiency (betalain-accumulating shoots).

FIGS. 11A-E show In-planta transformation and genome-editing of β-Lycopene cyclase 2 (βLCY2). (A) Schematic diagram of the βLCY2 role in the carotenoid biosynthesis pathway in plants. Lycopene β-cyclase2 (β-LCY2), a fruit chromoplasts-specific lycopene cyclase, facilitates the transition from lycopene to β-carotene. This transition leads to the loss of lycopene accumulation in chromoplasts and the gradual loss of red pigmentation in the fruit pulp. (B) Simplified schematic of the CRISPR/Cas9 vector designed for genome editing of the βLCY2 gene. The construct includes a constitutively expressed SpCas9, (C) a gRNA cassette targeting βLCY2 (comprising a pU6-sgRNA-scaffold-terminator unit and a tRNA-based multiplex editing module driven by the endogenous CsU6 promoter), an antibiotic resistance marker, a RUBY visual reporter, and a cassette expressing the IPT developmental regulator. (D) Regenerated transgenic shoot (#150-1) exhibiting pink pigmentation due to RUBY expression, indicating successful integration of the editing system. (E) Sequencing results from βLCY2-edited chimera #150-1, showing mutations at the gRNA3 target site (red box). A frameshift mutation in sequence 150-1 (blue box) results from an insertion, producing dual peaks downstream of the edit site. Additional mutations include a single-base ‘A’ insertion, a 1-bp deletion, and a large deletion in sequences 150-1C2, 150-1C3, and 150-1C4, respectively. SEQ ID Nos. 93-97 are indicated.

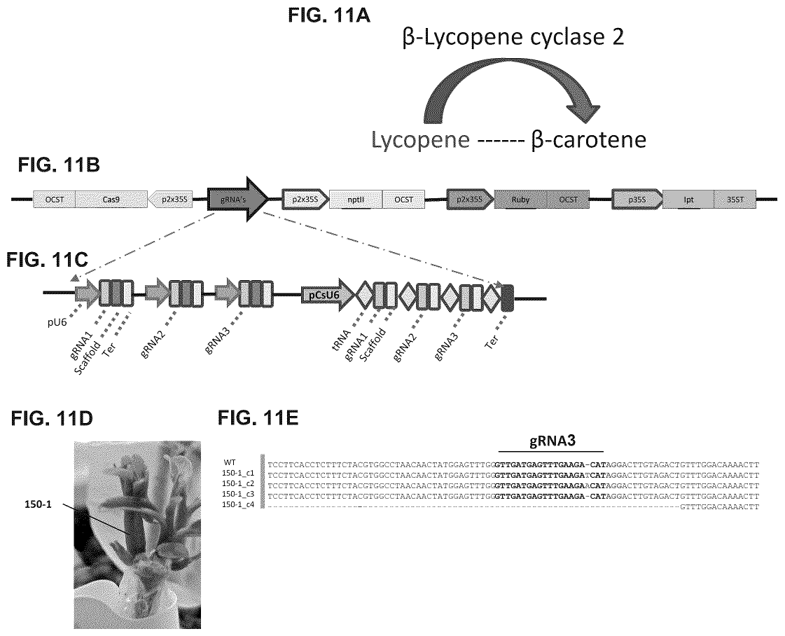

### DESCRIPTION OF SPECIFIC EMBODIMENTS OF THE INVENTION

The present invention, in some embodiments thereof, relates to nucleic acid constructs for genome editing.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As of today, the production of elite tree cultivars relies on the traditional cross-breeding practice, a lengthy process spanning up to a decade. Recent advances in the field of genome editing offer a promising avenue for precise trait improvements and accelerated breeding. However, the predominant method for genome editing in trees involves the time-consuming tissue-culture approach, thereby delaying the overall cultivation progress.

Whilst conceiving embodiments of the invention and reducing to practice, the present inventors designed a new tool for genome editing in plants and specifically in trees, more specifically in Citrus trees. Specifically, the present inventors introduce an in-planta approach for achieving genome editing in soil-grown plants such as Citrus plants, as a tree model, through direct transformation at the apical site of young seedlings. This genetic system, in some embodiments of the invention, is named CRISPRUS (a combination of CRISPR and Citrus) or In-Planta Genome-Editing in Citrus (IPGEC) and involves the simultaneous activation of diverse gene groups during Agrobacterium treatment: (1) a genome-editing reagents group, (2) a shoot induction and regeneration group, optionally (3) a gene for optimizing Agrobacterium T-DNA delivery, (4) and optionally an additional element dedicated specifically for preventing T-DNA integration to obtain transgene-free plants.

This integrated system significantly improved de novo shoot induction and regeneration efficiency. By incorporating single guides RNA's (sgRNA's), targeting the carotenoid biosynthetic gene PHYTOENE DESATURASE (CsPDS), the CRISPRUS system effectively produced mutated PDS-impaired shoots, displaying an albino phenotype, thereby confirming its ability to generate homozygous/bi-allelic genome-edited plants. Additional target gene was modified using thus approach beta lycopene cyclase 2. Moreover, transgene-free plants were obtained using a passive method (no selection) and an active method for preventing TDNA integration, e.g., by silencing of PolΘ.

Thus, an in-planta method to produce new edited organs such as from young tree (e.g., Citrus) seedlings is provided. The approach involves the combination of genome editing, developmental regulators, and optionally any of a specific meristem induction cassette, an enhanced Agrobacterium delivery system, and optionally a unit dedicated to preventing T-DNA integration; applied through a simultaneous expression system delivered by Agrobacterium directly to a wounded site of young seedlings. The present findings highlight the efficiency of the CRISPRUS system's components in inducing newly edited shoots, which can later be grafted to produce a new variety. Moreover, the present results demonstrate the system's capability to produce transgene-free, edited at T₀ plants, thus eliminating the need for a transgene disposal step in the next generation.

T₀ refers to a non-transgenic plant that has been subjected to the genome editing construct or system as described herein, has undergone editing, yet does not contain any foreign transgene in its genome. Its progeny are also non-transgenic.

In conclusion, this cost-effective and time-efficient approach to producing genome-edited plants paves the way for accelerating Citrus cultivation by enabling the development of Citrus plants with specific trait improvements. This method is applicable to other species and has the potential to promote the cultivation process of fruit trees in general.

Thus, according to an aspect of the invention there is provided a nucleic acid construct or construct system comprising:
(i) at least one nucleic acid sequence encoding a genome editing agent for introducing a sequence variation in a target gene of interest in a plant cell; and
(ii) at least one nucleic acid sequence encoding at least one gene being a developmental regulator. As used herein “a developmental regulator” is a gene product which is involved in meristem (e.g., axillary meristem) induction and/or regeneration.

Non-limiting examples are provided herein, however orthologs and homologs are contemplated herein, either naturally occurring or synthetic.
PLT1, AT3G20840
PLT2, AT1G51190
PLT4 (BABY BOOM, BBM) AT5G17430
PLT5, AINTEGUMENTA-LIKE 5 AT5G57390,
PLT3, AINTEGUMENTA-LIKE 6, AT5G10510
IPT3 AT3G63110
IPT2 AT2G27760
IPT4 AT4G24650
PLT7 AT5G65510
IPT9 AT5G20040
CUC1 AT3G15170
CUC2 AT5G53950
ESR1 AT1G12980
ESR2 AT1G24590
WIND1 AT1G78080
GRF4 AT3G52910
GIF1 AT5G28640
GRF5 AT3G13960
MONOPTEROS/ARF5 AT1G19850
WOX1 AT3G18010
WOX2 AT5G59340
WOX3 AT2G28610
WOX4 AT1G46480
WOX5 AT3G11260
WOX7 AT5G05770
WOX8 AT5G45980
WOX9 AT2G33880
WOX11 AT3G03660
WOX13 AT4G35550
LBD16 AT2G42430
LBD17 AT2G42440
LBD18 AT2G45420
LBD29 AT3G58190
ARF6 AT1G30330
ARF7 AT5G20730
ARF19 AT1G19220
LAX1 AT5G01240
LAS AT1G55580
RAX1 AT5G23000
EXB1 AT1G29860
ROX AT5G01305
ARR1 AT3G16857
ATXR2 AT3G21820
LDL3 AT4G16310
HTR15 AT5G12910
WUS AT2G17950
STM AT1G62360

According to a specific embodiment, the developmental regulator comprises a single or a plurality of genes, e.g., 2, 3, 4, 5, 6 and up to 10 genes.

According to a specific embodiment, the developmental regulator is selected from the group consisting of GRF4, IPT, GIF1, WUS, STM and PLT5.

According to some embodiments, the developmental regulator is selected from the group consisting of LAS, RAX1, EXB1, ROX and ARR1.

According to a specific embodiment the combination of developmental regulators comprises GRF4-GIF1 or WUS-STM.

According to some embodiments, the developmental regulator is IPT.

For overexpression, any of the nucleic acid sequence encoding a gene of interest disclosed and claimed herein can be the natural genes, or synthetic modifications such as cDNA thereof or homologs or orthologs thereof such as at least 60%, 70%, 75%, 80%, 90%, 95%, or more 96%, 97%, 98%, 99% identity to a nucleic acid sequence encoding the protein product of the gene, as long as their function is maintained e.g., developmental regulator.

Percent identity can be defined using alignment tools such as global (e.g., Needleman-Wunsch algorithm) or local alignment tools (e.g., Blast) which are well known in the art.

As used herein “a gene for facilitating Agrobacterium delivery into nuclei of the plant cells” also referred to herein as “a delivery-related gene” refers to a nucleic acid molecule which encodes an expression product capable of enhancing the delivery of genes (e.g., in the form of T-DNA) from the Agrobacterium into the nucleus of the plant cells

These can be bacterial (e.g., VirE2) or plant genes (e.g., VIP1) with further examples following.

Non-limiting examples are provided herein, however orthologs and homologs are contemplated herein, either naturally occurring or synthetic.

Bacterial (Agrobacterium tumefaciens) genetic factors to improve Agrobacterium delivery by plant expression:
VirE2 Gene ID: 86882444
VirE3 Gene ID: 86882422
VirD2 Gene ID: 86882437
VirD5
VirG Gene ID: 86882433
VirF Gene ID: 66841099

Plant genetic factors to improve Agrobacterium delivery by plant expression:
VIP1 AT1G43700
VIP2 AT5G59710
IMPα-4 A T1G09270
VBF AT1G56250
XRCC4 AT3G23100

According to a specific embodiment, the at least one gene for facilitating Agrobacterium delivery into nuclei of the plant cells comprises at least two genes, such as a nucleic acid molecule of a bacterial gene, e.g., VirE2 and a nucleic acid molecule of a plant gene, e.g., VIP1 or any homolog, ortholog as described hereinabove.

According to some embodiments, the gene used to facilitate nuclear import of the T-DNA complex into plant cell nuclei may be selected from a group consisting of importin β, histone proteins (such as H2A or H3), and engineered fusion constructs incorporating defined nuclear localization signals (NLSs). For example, a synthetic gene fusion may include a canonical NLS such as the SV40 large T-antigen NLS (PKKKRKV, SEQ ID NO: 1), which is widely used to direct proteins to the nucleus in plant and animal systems. Such NLS sequences may be fused to proteins involved in T-DNA transport—such as VirE2, VirD2, or plant-derived scaffold proteins—to enhance their nuclear localization independently of host factors like VIP1 or importins

As mentioned, the nucleic acid construct or construct system comprises at least one nucleic acid sequence encoding at least one genome editing agent for introducing a variation in a target gene of interest in a plant cell.

Embodiments of genome editing technologies include, but are not limited to engineered endonucleases such as meganucleases, zinc finger nucleases (ZFNs), transcription activator-like effector nucleases (TALENs), and CRISPR/Cas systems, which are applicable to a wide range of eukaryotic organisms, including plant cells. In the context of plant genome engineering, these systems enable precise, targeted modifications of nuclear DNA, including the generation of gene knockouts, insertions, and allele-specific edits. While originally developed or characterized in microbial or animal systems, these genome editing tools have been successfully adapted and optimized for use in a variety of plant species. The same double-strand break (DSB) repair mechanisms—non-homologous end joining (NHEJ) and homology-directed repair (HDR)—are conserved in plants and provide the foundation for applying these nuclease platforms in plant transformation workflows. Following is a non-limiting description of selected agents for genome editing in plants.

Genome Editing using engineered endonucleases—this approach refers to a reverse genetics method using artificially engineered nucleases to cut and create specific double-stranded breaks at a desired location(s) in the genome of plant cells, which are then repaired by cellular endogenous processes such as, homology directed repair (HDR) and non-homologous end-joining (NFfEJ). NFfEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. These editing mechanisms, while initially characterized in other systems, are conserved in plant cells and have been successfully applied to a wide range of plant species. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

These systems have been widely applied in the context of plant genome editing, including in major crop species.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This specificity has been leveraged for plant genome editing applications to generate targeted double-strand breaks at defined loci. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010). These have been widely applied in plant species for trait modification and functional genomics.

Basically, ZFNs and TALENs restriction endonuclease technology utilizes a non-specific DNA cutting enzyme which is linked to a specific DNA binding domain (either a series of zinc finger domains or TALE repeats, respectively). Typically, a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is FokI. Additionally, FokI has the advantage of requiring dimerization to have nuclease activity and this means the specificity increases dramatically as each nuclease partner recognizes a unique DNA sequence. To enhance this effect, FokI nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in plant cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in plant cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. TALEN can also be designed and obtained commercially from e.g., Sangamo Biosciences™ (Richmond, CA).

CRISPR-Cas system—The CRISPR/Cas genome editing system has been successfully and widely applied in plant systems. Many bacteria and archaea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded breaks in a variety of different species, including dicot and monocot plant species (Cho et al., 2013; Cong et al., 2013; DiCarlo et al., 2013; Hwang et al., 2013a,b; Jinek et al., 2013; Mali et al., 2013).

The CRISPR/Cas system for genome editing contains two distinct components: a gRNA and an endonuclease e.g., Cas9. The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded breaks produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA. A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes. However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the target gene. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publicly available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

Non-limiting examples of a gRNA that can be used in the present invention some of which are exemplified in the Examples section, showing effectivity on selected reporter genes such as PDS causin albine phenotype when the variation is successfully introduced, or of a target gene e.g., beta lycopene cyclase 2.

In order to use the CRISPR system, both gRNA and Cas9 should be expressed in a target cell.

Non-limiting examples of CAS enzymes:

Cas9 (D10A)
pcoCas9(hCas+intron)
https://www(dot)ncbi(dot)nlm(dot)nih(dot)gov/nucleotide/KF264451.1?report=genba
nk&log$=nuclalign&blast_rank=8&RID=7MYG7PKR016

Cas12, e.g., Cas12a, Cas12b
Cas13;
CasX.

According to some embodiments, the genome editing agent is at least one component of CRISPR, such as Cas9.

According to some embodiments, the nucleic acid construct or construct system comprises at least one genome editing agent. Thus for example one or more (e,g., 2, 3, 4, 5, or more say up to 10) copies of the endonuclease (e.g., CAS) can be included in the nucleic acid construct or construct system. As shown in Example 1a system which comprises 4 constructs includes 3 copies of Cas9.

According to some additional or alternative embodiments, the nucleic acid construct or construct system comprises at least one gRNA or more (e.g., 2, 3, 4, 5, 6, 7, 8, 9, 10 and up to 50) such as to different positions in the target genes [e.g., as shown in Example 1, gRNAs were expressed to 10 different exons included in the plasmid or system and referred to as “genome-editing booster”].

It will be appreciated that variation may also comprise an epigenetic variation. Hence the present teachings contemplate including in the construct(s) at least one coding sequence which is transcribed and optionally translated to an expression product which adds or removes an epigenetic modification preferably in a directed manner. Hence according to some embodiments, the genome editing agent may form a complex with such a heterologous coding sequence (such as an in-frame fusion).

According to a specific embodiment, the epigenetic editing agent is a demethylase enzyme that can be in-frame fused to an editing nuclease such as one from the CRISPR system to form a fusion such as CRISPR/dCas9-demethylase or any other enzyme, e.g., TET1. This construct enables targeted demethylation of specific promoter regions in plant genomes, leading to transcriptional activation without altering the underlying DNA sequence. In Arabidopsis thaliana, a dCas9-SunTag-TET1 fusion was used to demethylate the promoter of the FWA gene, resulting in stable activation of gene expression and a late-flowering phenotype. Importantly, the induced epigenetic state was at least partially heritable across generations, demonstrating the potential of such tools for long-term gene regulation in plants (Papikian et al., 2019, Scientific Reports, 9, 11960, incorporated herein by reference). Other nuclease-based epigenetic systems can include but not limited to demethylases like TET1, histone acetyltransferases, and DNA methyltransferases (Wang et al., 2022, International Journal of Biological Macromolecules, 222, 2233-2243; Zhang et al., 2021, Journal of Genetics, 100, 12, each of which is incorporated herein by reference in its entirety).

As used herein sequence variation may cause a loss of function in the target gene of interest.

As used herein, the phrase “loss-of-function alterations” refers to any mutation in the DNA sequence of a gene, which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the regulatory activity of the protein; a nonsense mutation, i.e., a mutation which introduces a stop codon in a protein, e.g., an early stop codon which results in a shorter protein devoid of the regulatory activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the regulatory activity), or in a longer amino acid sequence (e.g., a readthrough protein) which affects the secondary or tertiary structure of the protein and results in a non-functional protein, devoid of the regulatory activity of the non-mutated polypeptide; a readthrough mutation due to a frame-shift mutation or a modified stop codon mutation (i.e., when the stop codon is mutated into an amino acid codon), with an abolished regulatory activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the target gene may be in a homozygous form or in a heterozygous form. According to this embodiment, homozygosity is a condition where both alleles are characterized by the same nucleotide sequence.

According to a specific embodiment the loss of function mutation is in both alleles in a homozygous or heterozygous form yet both alleles encode for dis-functioning products, also referred to biallelic loss-of-function or biallelic inactivation.

According to other embodiments, the variation causes a gain of function.

As used herein, the phrase “gain-of-function alterations” refers to any mutation in the DNA sequence of a gene that results in upregulation of expression and/or enhancement or change of function of the expressed product, i.e., the mRNA transcript and/or the translated protein. Non-limiting examples of such gain-of-function alterations include missense mutations that increase the activity or confer a new activity on the protein; promoter mutations leading to increased gene expression; duplications or insertions that result in overexpression; and fusion events or truncations that remove inhibitory domains or regulatory elements, thereby enhancing activity or altering substrate specificity. According to specific embodiments, gain-of-function alterations may affect one or both alleles of the gene and may occur in homozygous or heterozygous form. In cases where both alleles produce a hyperactive or novel-function product, this is referred to as biallelic gain-of-function.

According to a specific embodiment, the nucleic acid construct, further comprises (iv) at least one nucleic acid sequence for preventing T-DNA integration to the plant cell genome. Such a gene is also referred to as a “gene associated with T-DNA integration”.

As used herein, a “gene associated with T-DNA integration” refers to any endogenous plant gene whose expression or activity facilitates, promotes, or is otherwise required for the stable incorporation of transferred DNA (T-DNA) into the plant genome following Agrobacterium-mediated transformation. According to some embodiments, such genes may function in DNA repair, chromatin remodeling, T-DNA tethering, or related processes that enable the physical joining of T-DNA with host genomic DNA.

According to some embodiments, the sequence is directed against DNA Polymerase θ (Polθ, also known as POLQ), a key enzyme in the alternative end-joining (alt-EJ) pathway responsible for the majority of T-DNA integration events in plant cells.

In other or additional embodiments, integration prevention may be achieved through targeted modulation of other host factors involved in non-homologous end joining (NHEJ) or T-DNA tethering. For example, suppression of KU70, KU80, or DNA Ligase IV can reduce canonical NHEJ activity and thus limit T-DNA insertion at double-strand breaks.

Methods for down-regulating expression or activity of a gene of interest are well known in the art.

As used herein the phrase “dowregulates expression” refers to dowregulating the expression of a protein (e.g. DNA Polymerase θ) at the genomic (e.g. homologous recombination and site specific endonucleases) and/or the transcript level using a variety of molecules which interfere with transcription and/or translation (e.g., RNA silencing agents) or on the protein level (e.g., small molecules and inhibitory peptides, antagonists and the like).

For the same conditions the expression is generally expressed in comparison to the expression in a cell of the same species but not contacted with the agent or contacted with a vehicle control, also referred to as control.

Down regulation of expression may be either transient or permanent.

According to specific embodiments, down regulating expression refers to the absence of mRNA and/or protein, as detected by RT-PCR or Western blot, respectively.

According to other specific embodiments down regulating expression refers to a decrease in the level of mRNA and/or protein, as detected by RT-PCR or Western blot, respectively. The reduction may be by at least a 10%, at least 20%, at least 30%, at least 40%, at least 50%, at least 60%, at least 70%, at least 80%, at least 90%, at least 95% or at least 99% reduction.

Non-limiting examples of agents capable of down regulating expression of at least one gene associated with T-DNA integration (e.g., DNA Polymerase θ) are described in details hereinbelow.

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

According to specific embodiments, the downregulating agent is a polynucleotide.

According to specific embodiments, the downregulating agent is a polynucleotide capable of hybridizing to a gene or mRNA encoding at least one gene associated with T-DNA integration (e.g., DNA Polymerase θ).

According to specific embodiments, the downregulating agent directly interacts with at least one gene associated with T-DNA integration (e.g., DNA Polymerase θ).

According to specific embodiments, the agent directly binds at least one gene associated with T-DNA integration (e.g., DNA Polymerase θ).

According to specific embodiments, the agent indirectly binds at least one gene associated with T-DNA integration (e.g., DNA Polymerase θ), e.g. binds an effector of the gene.

Thus, according to some embodiments, downregulation of at least one gene associated with T-DNA integration (e.g., DNA Polymerase θ) can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., DNA Polymerase θ) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA.

Design of antisense molecules which can be used to efficiently downregulate a at least one gene associated with T-DNA integration (e.g., DNA Polymerase θ) must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

Algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Downregulation of at least one gene associated with T-DNA integration (e.g., DNA Polymerase θ) can also be achieved by inactivating the gene (e.g., DNA Polymerase θ) via introducing targeted mutations involving loss-of function alterations (e.g. point mutations, deletions and insertions) in the gene structure. Such methods are well known in the art and include also genome editing as described above.

According to a specific embodiment, down regulation in the gene associated with T-DNA integration to the plant genome e.g., DNA Polymerase θ is effected in a transient manner.

According to other embodiments, the plant is selected non-transgenic yet mutated in such a gene associated with T-DNA integration to the plant genome e.g., DNA Polymerase θ. Such a plant can be subjected to the nucleic acid construct or construct system

As used herein “nucleic acid construct” refers to a nucleic acid

Each of the elements e.g., (i), (ii), (iii) and/or (iv) can be on the same construct and/or different constructs. For example, component (iii) i.e., at least one nucleic acid sequence which encodes at least one gene for facilitating Agrobacterium delivery into the nuclei of plant cells may be on one construct such as construct 409 while component (ii): developmental regulators can be on different constructs such as in 501 (WUS-STM) and 502 (ipt). Promoters that are active in dividing and differentiating cells:

pYAO—This promoter is active in tissues that are active in cell division, including shoot apex (meristematic tissue). YAO is also active in processes such as embryogenesis and gametogenesis—it creates some unidentified “RESTART” for the plant potent cells. It has very low expression in non-dividing tissues, such as leaves and petioles.

CmYLCV The CmYLCV promoter is highly active in callus, meristems and vegetative and reproductive tissues.

Non-limiting examples of Polymerase targets are provided in Table 1 below.

TABLE 1

| | | |
|---|---|---|
| Pol α | POLA1 | AT5G67100 |
| | POLA2 | AT1G67630 |
| Pol δ | POLD1 | AT5G63960 |
| | POLD2 | AT2G42120 |
| | POLD3 | AT1G78650 |
| | POLD4 | AT1G09815 |
| Pol ε | POL2A | AT1G08260 |
| | DPB2 | AT5G22110 |
| Pol zeta | REV3 | AT1G67500 |
| | REV7 | AT1G16590 |
| Pol lambda | POL lambda | AT1G10520 |
| Pol eta | POLH | AT5G44740 |
| Pol kappa | POL kappa | AT1G49980 |
| Pol Rev1 | POL Rev1 | AT5G44750 |
| Pol theta | POLQ | AT4G32700 |
| Pol1-like A | POL Gamma1 | AT3G20540 |
| Pol1-like B | POL Gamma2 | AT1G50840 |

The system or nucleic acid construct may also include a gene for selection. Selection may be applied for a very short time or using a low concentration of the selecting agent, weak promoters, developmental promoter (active in non-differentiated cells) and/or low copy number. It is suggested that such a mode of expression prioritizes the cells that transiently express the construct or construct system, with the most undifferentiated cells expressing first and expression is lost through development.

Target genes can be those selected for conferring an agriculturally valuable trait, such as yield, vigor, increased nitrogen use efficiency, resistance or tolerance to biotic or abiotic stress.

Non-limiting examples are provided in Table 2 below which may be of relevance to many plants including trees, e.g., Citrus:

TABLE 2

| Gene target for genome editing | Gene Identifiers in Citrus |
|---|---|
| Lycopene beta cyclase 2 | UZH23304.1 |
| 2OGD | Cg2g000710 |
| 1,2 rhamnosyltransferase | Q8GVE3.2 |
| AGL6 | XP_006490967.1 |
| AGL11 | KAH9781294.1 |
| Della | KAH9739056.1 |

As used herein, a “nucleic acid construct” or “vector” refers to an artificially assembled or recombinant nucleic acid molecule, which may be DNA or RNA, single-stranded or double-stranded, linear or circular, that is designed to achieve a defined biological function when introduced into a host cell, e.g., gain of function or loss of function. A nucleic acid construct typically comprises at least one coding sequence (e.g., a gene of interest and/or a down-regulating agent such as described above), and may further include one or more regulatory or functional elements such as cis-acting regulatory elements such as promoters, enhancers, terminators, untranslated regions (UTRs), origins of replication, or signal peptides, as well as selectable markers, depending on the intended use.

As used herein “a nucleic acid construct system” comprises a plurality of nucleic acid constructs typically aimed at achieving a shared function (e.g., in planta non-transgenic genome editing by meristem induction). In this case, for example, one which encodes at least one developmental regulator and another which encodes a genome editing agent such as Cas9.

However, this division is not to be taken as restrictive as a genome editing component may still be presented on the construct of the construct system which encodes the developmental regulator. For instance, as exemplified herein, the combination of 501 and/or 502, 216 and optionally 409.

In certain embodiments, the nucleic acid construct comprises a T-DNA vector suitable for Agrobacterium-mediated transformation.

According to some embodiments, the vector comprises a T-DNA region flanked by right border (RB) and left border (LB) sequences, which define the boundaries of the DNA segment to be transferred into the plant cell. According to some embodiments, one or more coding regions and regulatory elements are comprised in a single RB/LB define segment.

According to some embodiments, the T-DNA vector comprises at least one coding sequences, which may encode a protein of interest (e.g., developmental regulator, a gene for facilitating Agrobacterium delivery and/or a genome editing agent e.g., Cas9) or a nucleic acid agent such as gRNA and/or RNA silencing agent.

According to some embodiments, the coding agent is operably linked to at least one plant-compatible cis-acting regulatory element such as a promoter, which may be constitutive (e.g., Cauliflower Mosaic Virus 35S promoter), tissue-specific (e.g., root-specific or seed-specific promoters), developmentally regulated, inducible, or synthetic.

According to a specific embodiment, each coding sequence can be under the transcriptional regulation of a cis-acting regulatory element of the same type (e.g., 35S) or of different types (e.g., 35S and Ubi10, pNOS).

According to some embodiments, the guide RNAs may be expressed from RNA polymerase III promoters such as U6 or U3 promoters of plant origin.

According to some embodiments, at least one regulatory element drives transcription of a coding region (e.g., 1, 2, or more, e.g., p2X35S which comprises a double enhancer version of 35S.

According to a specific embodiment, the promoter is active in cells undergoing cell division.

According to some embodiments, the promoter is active in dividing or meristematic plant cells, such as those found in embryos, meristems, or regenerating tissues. Promoters with cell division-specific activity may include, for example, the Arabidopsis YAO promoter (as exemplified herein in construct 409), which drives expression in the zygote, embryo, and shoot/root meristems. According to some embodiments, the RPS5a promoter from Arabidopsis is another example. Other suitable promoters include, but are not limited to, the Arabidopsis EC1.2 or EC1.1 promoters, which are active specifically in the egg cell and are used to restrict genome editing to the germline, allowing generation of non-mosaic, heritable mutations. The CYCB1 and CDC2A promoters, derived from Arabidopsis or rice, are regulated by the cell cycle and are active during specific phases such as G2/M or S phase, making them useful for targeting actively dividing cells. Additionally, general constitutive promoters that show enhanced activity in dividing cells may be used. These include plant ubiquitin promoters (e.g., maize Ubiquitin1), which drive broad and robust expression across many tissues but are particularly strong in metabolically active and dividing cells. The ACTIN promoter, while not exclusive to dividing cells, also provides consistent expression in structural and growing tissues. In some embodiments, promoters derived from developmental regulators e.g., BBM) or WUS (embodiments of which are described in details above) may be used.

Without being bound by theory it is suggested that the use of promoter(s) active in dividing cells would enable more uniform genetic changes throughout the developing plant tissue.

In certain embodiments, the regulatory element e.g., promoter, is derived from a plant, viral, bacterial, or synthetic origin.

According to some embodiments, the coding sequence is operably linked to a transcriptional terminator or polyadenylation signal, such as the NOS terminator, OCS terminator, or CaMV 35S poly(A) signal, which facilitates proper transcript processing and stability.

According to some embodiments, the nucleic acid construct further comprises at least one selectable marker gene to enable selection of transformed cells. Suitable markers include, but are not limited to, nptII (conferring kanamycin resistance, as exemplified in construct 501), bar (conferring phosphinothricin resistance), hpt (conferring hygromycin resistance), or visually screenable markers such as GUS, GFP, or mCherry.

According to some embodiments, the nucleic acid construct(s) may include one or more reporter genes. Reporter genes may be used to assess transformation efficiency or spatial and temporal expression of the construct.

According to some embodiments, the nucleic acid construct(s) includes introns and untranslated regions (UTRs), such as a 5′ UTR or 3′ UTR, to enhance transgene expression through mechanisms such as intron-mediated enhancement (IME) or improved mRNA stability. Suitable examples include the maize ubiquitin 1 intron or the Arabidopsis EF1α 5′ UTR.

According to some embodiments, the nucleic acid construct(s) further comprises elements derived from plant or viral genomes, such as translational enhancers (e.g., TMV omega leader sequence), replication origins (e.g., BeYDV replicons), or movement proteins to facilitate intracellular or systemic transport of the expressed molecule.

According to some embodiments, the nucleic acid construct(s) encodes multiple proteins from a single transcript using mechanisms such as viral 2A peptides, self-cleaving ribozymes, or internal ribosome entry sites (IRES), thereby enabling polycistronic expression from a single promoter.

According to some embodiments, the T-DNA vector is used in conjunction with systems that minimize or prevent stable genomic integration of the transferred DNA, thereby promoting transient expression. For example, the vector may incorporate virus-derived replicons, such as Bean yellow dwarf virus (BeYDV) or Wheat dwarf virus (WDV) replication elements, which enable episomal maintenance of the T-DNA in plant nuclei.

According to some embodiments, elements located outside the T-DNA borders include one or more origins of replication (e.g., pUC, ColE1, RK2, pVS1) for propagation in Escherichia coli and Agrobacterium tumefaciens, and antibiotic resistance markers for bacterial selection (e.g., spectinomycin, carbenicillin, kanamycin).

According to an aspect of the invention, there is provided a method of producing a genome edited plant or part thereof comprising contacting the plant or part thereof with the nucleic acid construct or construct system as described herein under conditions which allow editing and regeneration.

According to some embodiments, the nucleic acid construct(s) is introduced into Agrobacterium tumefaciens strain or strains. The introduction can be by any method known in the art such as by electroporation, freeze-thaw transformation, or conjugation. The resulting Agrobacterium strain(s), harboring the T-DNA construct, is then brought into contact with a plan for in planta transformation, under conditions suitable for T-DNA transfer.

As used herein, the term “in planta transformation” refers to a method of delivering a nucleic acid construct, such as a T-DNA vector, directly into a plant or an intact part of a plant, typically without the use of isolated cells, callus culture, or tissue regeneration steps.

In some embodiments, the plant part comprises a leaf disc, cotyledon, hypocotyl, root, shoot apical meristem, somatic embryo, or embryonic callus.

In other embodiments, the plant is exposed to Agrobacterium, for example by injection, immersion (dipping) or via vacuum infiltration or floral dip, enabling transformation of internal tissues comprising meristematic cells.

According to some embodiments prior to contacting with the Agrobacterium, the plant is wounded, such as by cutting, for example, in seedlings, above cotyledons and under the first true leaf.

According to some embodiments, the wounded shoot apical meristem is exposed to at least one Agrobacterium strain harboring the T-DNA construct(s).

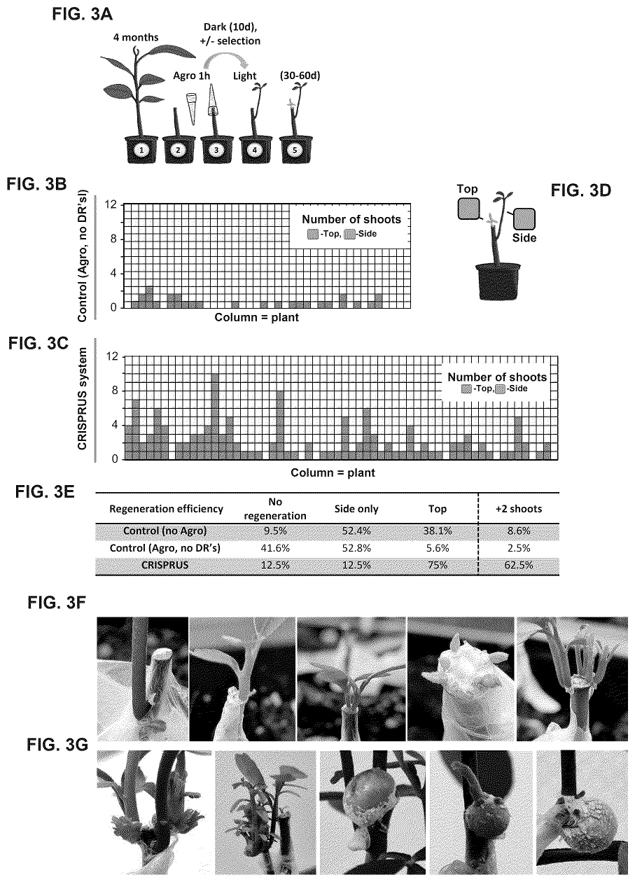

An exemplary method of in planta transformation is described in FIG. 3A.

According to some embodiments, the conditions comprise exposing to blue light prior to said contacting such as for 12-24 hours. It is suggested that blue light pre-exposure may be beneficial for improved regeneration as shown (Dong et al., 2023).

According to some embodiments, the conditions comprise selection for a short period of time (e.g., 2-10 days, e.g., 2-3, 2-4, 2-5, 2-6, 2-7, 2-8, 2-9, 3-4, 3-5, 3-6, 3-7, 3-8 or 3-9) and/or using low concentration of any of (i), (ii), (iii) and/or (iv), to ensure transfer of the T-DNA but avoid integration to the plant genome. The selection conditions depend on the type of the selection gene.

According to some embodiments, the conditions comprise maintaining the plant or part thereof in the dark such as for 7-14 days (e.g., 8-12 days, e.g., 10 days) to promote regeneration following contacting with the bacteria and prior to exposing to light.

According to some embodiments, the plant is then transferred to light for a time period of 30 to 60 days.

According to some embodiments and without being bound by theory, the dark incubation step enhances T-DNA delivery and transformation efficiency through several mechanisms. In particular, darkness has been shown to increase plant cell competence by reducing light-induced oxidative stress and by modulating hormonal balance and wound responses, which can enhance cell division and receptivity to transformation. Additionally, virulence (vir) gene expression in Agrobacterium is optimized at moderate temperatures and low-light or dark conditions, particularly when induced by phenolic compounds such as acetosyringone.

According to some embodiments, after the dark incubation period, the treated plant material is transferred to light conditions to support subsequent growth, regeneration, or selection. Light exposure is necessary for the recovery and proliferation of transformed cells, particularly in green tissues that require photosynthetically active radiation. Thus, the sequential use of dark followed by light incubation provides a favorable physiological environment for both Agrobacterium-mediated T-DNA transfer and plant tissue regeneration.

Validation of genome editing can be done at the DNA, RNA and/or protein level.

According to some embodiments, the presence of a genome editing event in a plant or plant part is validated using one or more molecular and biochemical methods. In certain embodiments, targeted sequence modifications, such as insertions, deletions, or base substitutions, are detected by amplifying the edited locus via sequencing or RT-PCR, high-throughput sequencing (e.g., Illumina), T7E1 mismatch cleavage assay, or restriction fragment length polymorphism (RFLP) analysis if the edit disrupts or introduces a restriction site. In some embodiments, high-resolution melting (HRM) analysis is employed to identify heteroduplex DNA indicative of editing events.

According to some embodiments, the functional consequence of the editing is validated by phenotypic or biochemical assays that reveal a gain or loss of gene function. For example, in cases where a gene has been edited to disrupt enzymatic activity, protein expression or enzymatic function may be assessed via in situ activity assays, in vitro enzymatic activity measurements, or metabolic profiling of pathway outputs. Conversely, in cases involving gain-of-function edits or promoter swaps, upregulation of gene activity may be measured by enzyme-linked immunosorbent assay (ELISA), Western blot, immunohistochemistry, or substrate-specific colorimetric or fluorometric assays. In some embodiments, physiological or morphological traits associated with the gene of interest are evaluated to confirm the functional outcome of the editing (e.g., altered leaf morphology, pigment accumulation, or pathogen resistance). When a reporter gene is present in the system it can also be used to validate.

In certain embodiments, the plant or plant tissue is also analyzed to confirm the absence of stably integrated editing machinery, such as Cas9 or guide RNA expression cassettes, and/or any of the DR, gene facilitating transfer to the nucleus, reporter gene and any other gene which may render the plant transgenic, thereby demonstrating a non-transgenic status. This may be performed by PCR using primers specific to the coding region (e.g., guide RNA scaffold sequence and/or Cas9). In some embodiments, sequencing, RT-PCR or Northern blot is employed to confirm the absence of corresponding transcripts. In other embodiments, protein-level detection methods such as ELISA or Western blot are applied such as to verify the absence of Cas9 protein expression. Where the editing machinery was transiently delivered (e.g., as RNPs or mRNA), and no T-DNA integration occurred, no corresponding nucleic acid or protein signal is expected in the analyzed tissue.

According to some embodiments, genome-wide expression profiling (e.g., RNA-seq or microarray analysis) may be performed to evaluate off-target transcriptional effects or confirm the specific activation or suppression of the targeted gene(s). These methods may be complemented by RNA in situ hybridization or in situ RT-PCR to spatially resolve transcript accumulation in specific tissues or cell types. According to some embodiments, once the genome-edited plant part has been validated for the desired genetic modification and optionally confirmed to be free of stably integrated editing components (e.g., Cas9 or guide RNA sequences) or other transgene(s), it is used as grafting material. In certain embodiments, the genome-edited shoot is grafted onto a mature, e.g., flowering-capable scion or rootstock of the same or a compatible species. The grafting process enables accelerated development of reproductive tissues (e.g., flowers or fruit), thereby shortening the overall breeding cycle and facilitating earlier generation of edited progeny.

In some embodiments, the edited shoot contains meristematic or juvenile tissue, and is grafted using wedge, cleft, or side-graft techniques under sterile or greenhouse conditions. The graft union is maintained using mechanical supports (e.g., clips or wraps), and the plant is grown under conditions favorable for vascular reconnection. In certain embodiments, the mature recipient plant provides the necessary hormonal and physiological signals to support rapid growth and floral induction in the edited tissue, even when the edited material would not flower on its own. This approach enables the propagation and evaluation of edited traits without the need for full plant regeneration, and can significantly reduce the time required to generate and test stable, edited lines.

As used herein, the term “compatible species” refers to a plant species that is physiologically and developmentally capable of forming a stable graft union with another species, allowing for vascular connectivity, nutrient exchange, and sustained growth of the grafted tissues. Compatibility is typically determined by taxonomic proximity (e.g., same genus or family), shared anatomical and biochemical traits, and established use in horticultural grafting practices.

According to some embodiments, the genome-edited shoot is grafted onto a mature plant of a compatible species or hybrid rootstock within the Citrus genus or closely related genera. For example, a genome-edited shoot derived from a commercially important Citrus variety such as Carrizo citrange (a hybrid of Citrus sinensis x Poncirus trifoliata), Duncan grapefruit (Citrus paradisi), Foster grapefruit, or Hudson grapefruit may be grafted onto a mature, flowering-capable rootstock or scion of a compatible Citrus species, including Citrus reticulata (mandarin), Citrus limon (lemon), or other widely used Citrus genotypes. These edited genotypes are selected for their agronomic relevance, and grafting onto mature compatible plants facilitates rapid floral induction and fruit development. This approach allows for early evaluation of the edited traits and significantly reduces the breeding cycle compared to conventional propagation or full regeneration from tissue culture. In such embodiments, the mature compatible plant provides physiological support, including hormonal signals, that facilitate early flowering and fruiting of the edited tissue, thereby enabling rapid evaluation and propagation of the desired genetic traits.

Examples of plants which can be subject to the present teachings include, but are not limited to trees/perennials, annual plants that are recalcitrant to genetic transformation as well as in combination with existing methods for propagation via tissue culture in monocots

Cannabis species. Metabolic engineering by genome editing is expected to be a major tool to develop lines of cannabis specializing in one or two major cannabinoids. While THC and CBD are currently the most abundant cannabinoids and have been extensively studied regarding their therapeutic use, approximately 80 other cannabinoids have not been studied and present a bank of potentially important compounds for medical use.

Capsicum species (bell peppers, chili peppers and others). Important crop plants for fresh use and for the production of natural food colors and spices.

Legume species (proviso transformable soybean). These important crop plants contain proteins rich in the nine “essential amino acids” and are essential ingredients in any plant-based diet.

Cucurbit species (melon, watermelon, pumpkin, squash, gourd, cucumber).

Examples of high value monocot plant species that would greatly benefit from the availability of a genome editing system.

Various palm species (date palm, coconut, oil palm)

Musa species (Banana).

Additional examples include species from the Citrus genus such as Citrus aurantium (bitter orange), Citrus maxima (pummelo), and Citrus junos (yuzu), as well as rootstock varieties such as Swingle citrumelo and Volkamer lemon. From the Malus genus: Malus domestica (apple). From the Pyrus genus: Pyrus communis (European pear), Pyrus pyrifolia (Asian pear). From the Prunus genus: Prunus persica (peach), Prunus armeniaca (apricot), Prunus domestica (plum), Prunus salicina (Japanese plum), Prunus avium (sweet cherry), Prunus cerasus (sour cherry), Prunus dulcis (almond). From the Juglans genus: Juglans regia (English walnut), Juglans nigra (black walnut). From the Carya genus: Carya illinoinensis (pecan). From the Vitis genus: Vitis vinifera (grapevine). From the Diospyros genus: Diospyros kaki (persimmon). From the Ficus genus: Ficus carica (fig). From the Punica genus: Punica granatum (pomegranate). From the Actinidia genus: Actinidia deliciosa, Actinidia chinensis (kiwifruit). From the Mangifera genus: Mangifera indica (mango). From the Litchi genus: Litchi chinensis (lychee). From the Annona genus: Annona cherimola (cherimoya), Annona squamosa (sugar apple). From the Tamarindus genus: Tamarindus indica (tamarind). From the Corylus genus: Corylus avellana (hazelnut).

Any of the edited plants can be processed to obtain a processed (edible or non-edible) product which comprises the genome edited DNA.

Processed Citrus products that may contain DNA include edible items such as in the case of Citrus, Citrus juices (including orange, lemon, and grapefruit juice), Citrus pulp, purees, segments, marmalades, jams, preserves, candied peels, Citrus zest or peel powders, and Citrus-based sauces and dressings containing fruit particles. Non-edible products that may contain DNA include in the case of Citrus, Citrus fiber powders derived from whole peel or pulp and Citrus-derived pectin preparations that retain some cellular residue. Highly purified products such as Citrus essential oils, seed oils, and distilled extracts generally do not contain detectable DNA.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Methods

### Plant Material and Growth Conditions

Plants used in this study were Carrizo citrange (Carizzo, Citrus sinensis ‘Washington’ sweet orange x Poncirus trifoliate), Duncan grapefruit (Citrus paradisi Macf.) or the Nicotianana benthamiana, as indicated in each experiment. Plants were grown in a 16 h light/8 h dark photoperiod at 25° C., 70 mol m⁻² s⁻¹ light intensity, in a soil potting mix containing (w/w) 70% peat, 30% perlite, supplemented with slow release fertilizer (Even-Ari, Israel).

### Plasmid Construction

The vectors used in this study were assembled using the Golden braid (GB) modular cloning system (Sarrion-Perdigones et al., 2013). Transcriptional units were synthesized by GeneArt (Invitrogene, https://www(dot)thermofisher(dot)com/il/en/home/brands/invitrogen(dot)html) and served for GB cloning. The GB assembly was conducted according to the GB protocol using the T4 ligase, and either BsaI-HF or BsmBI enzymes (New England Biolabs, https://www(dot)neb(dot)com/en). All vectors were inserted into Escherichia coli (DH5α)-heat-shock competent cells and selected using suitable antibiotics and X-gal, verified by PCR and Sanger sequencing. Electrocompetent Agrobacterium tumefaciens (EHA105 strain) was transformed using 100 ng plasmid by heat shock.

### Agrobacterium Infiltration for Transient Expression and Fluorescence

For transient expression assay, 8-months-old Carrizo plants were used. It will be appreciated that each Agrobacterium (agro) contains only one of the four plasmids, and grown in an independent tube. On the morning of the experiment, the different agros are mixed and applied together. Specifically, two agro populations harboring the 502 and 216 plasmids at OD₆₀₀=0.2 each, were mixed and immersed in infiltration medium that contained 10 mM MES [2-(N-Morpholino) ethanesulfonic acid sodium salt, Duchefa], 10 mM Mgcl₂, 5% sucrose, 200 μM acetosyringone (pH 5.6). Following 1 h at 28° C., 220 rpm, Agro were injected into the leaves using 1 ml syringe (Artsana group, Italy). Six plants, 3 leaves each were injected and samples were collected at three time points: 3, 6 and 10 days following infiltration, snap frozen in liquid nitrogen, and later used for expression analysis. At the end of the experiment, samples were taken also for confocal imaging.

### In Planta Transformation and Genome Editing of Citrus Seedlings

In-planta transformation onto Citrus seedlings was conducted following the work of (Zhang et al., 2017b, Rizwan et al., 2021) with slight modifications. Agrobacterium strain EHA105 harboring either the 501, 502, 216, or the 409 plasmids were grown in 50 ml LB with 50 mg l⁻¹ kanamycin (Kan; Duchefa) over night at 28° C. On the morning of the experiment, the different agros were mixed to a final OD₆₀₀ of 1.15, as follow: 0.35 for 501, 0.35 for 502, 0.25 for 216, and 0.2 for 409 (Agro-mix). The Agro-mix was centrifuged at 4000 rpm for 10 minutes and the cell pellet was resuspended with MS+5% Suc solution; a Murashige and Skoog (MS; Duchefa Biochemie, The Netherlands) medium was supplemented with 5% sucrose (Suc; Duchefa) and acetosyringone 200 μM (AS; Sigma-Aldrich, Israel, pH 5.8), kept for 2 h in 28° C., 220 rpm prior to the experiment. The epicotyls of a 4-months-old Carrizo or Duncan grapefruit seedlings were cut 4-5 cm above ground (above cotyledons and under the first true leaf) and a 10 μl tip filled with agro was placed on the decapitated site for 1 hour to induce infection (FIG. 3B). Following tip removal, the wound sites were wrapped with parafilm and kept in the dark for 10 days. Three days following co-culturing (plant-agro incubation), parafilm was removed and cotton balls submerged in agar trap solution (0.5MSx 0.5Suc, pH 5.8, 0.4% plant agar), containing 50 mg l⁻¹ Kan and 500 mg l⁻¹ Claforan (Cla; Cefotaxim, Duchefa), were used to immerse the wound sites at least two times. Wound sites with a remaining agar trap solution drops wrapped again with parafilm and remained in the dark for another 7 days. Then, plants were transferred to the light (70 mol m⁻² s⁻¹), kept in high humidity using a plastic domes covers. The induction of new shoots was notable from 3 weeks after infection and up to 3 months.

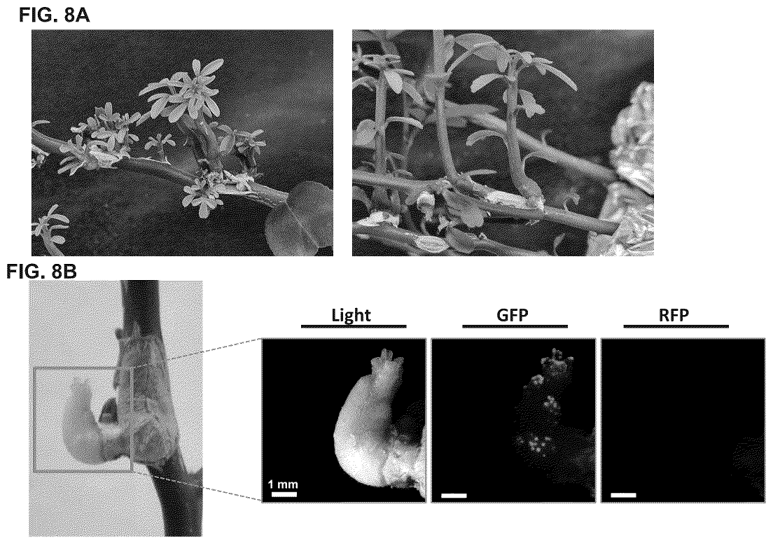

For the in-planta transformation using the vacuum method (FIGS. 8A-B), the apical, as well as the axillary meristems of 8-moths-old Carizzo plants were all removed. The plants were then dipped in a 400 ml Agro-mix infiltration medium with similar OD as described above and vacuum was applied for 4 min. Plants were then placed vertically in the dark for 10 days, later shifted to the light while maintained in high humidity.

RNA Extraction, cDNA Preparation and Quantitative Real-Time PCR
RNA was extracted from leaves or fruits using the Total RNA Mini Kit (Plant) from Geneaid (New Taipei, Taiwan). The success of the extraction and the integrity of the RNA were validated using NanoDrop (MaestroNano, New Taipei, Taiwan) and gel electrophoresis. cDNA strands were synthesized using a designated kit (qScript cDNA Synthesis Kit; QuantaBio, Beverly, MA, USA) and PCR (SimpliAmp™ Thermal Cycler; ThermoFisher Scientific, Waltham, MA, USA), according to the manufacturer's instructions. Quantitative PCR was performed using the PikoReal 96 Real-Time PCR System (Thermo Scientific, MA, USA) and qPCRBIO SyGreen Blue Mix Hi-ROX kit by PCR Biosystems (London, UK).

### Confocal Microscopy Imaging

Image acquisition was done using a Leica SP8 laser scanning microscope (Leica, Wetzlar, Germany) equipped with a solid-state laser with 488 nm light, a HC PL APO CS 63×/1.2 water immersion objective (Leica) and Leica Application Suite X software (LASX, Leica). Images of GFP signal were acquired using the 488-nm laser light and the emission was detected with HyD (hybrid) detector in a range of 500-525 nm. Autofluorescence of the chloroplasts was detected in a range of 650-750 nm with a PMT detector.

### Regeneration Efficiency Measurements

Regeneration efficiency was assayed 2 months following transformation. The number of shoots emerging from the side and those emerged from the top was counted for each plant. Regeneration efficiency was determined as the number of plants having at least one shoot emerging from the top out of the entire plants assayed. In addition, the percentage of plants having two shoots or more that have regenerated from the top was also determined.

TABLE 3
Primers used in this study for genotyping.
Adapter for Fwd. primer; Adapter for Rev primer.

| | Target | Primer sequence (5′-3′) | SEQ ID NO: |
|---|---|---|---|
| Large deletions | CsPDSEx1 F | TGCGATATGGTTTCCGAGAT | 2 |
| | CsPDSEx13 R | GCAGCAAGCAGCACATAGTC | 3 |
| NGS (a) | Ex1 gRNA1 NGSF | ACACTCTTTCCCTACACGACGCTCTTCCGATCTCC | 4 |
| | | GAGATAGTGAACCGATGG | |
| | Ex1 NGSR | GTGACTGGAGTTCAGACGTGTGCTCTTCCGATCTGGT | 5 |
| | | AGTCCACACAAACCACCT | |
| | Ex2 gRNA2 NGS | ACACTCTTTCCCTACACGACGCTCTTCCGATCTGG | 6 |
| | 2F | AAGCTGCTTACTTGTCTTCG | |
| | Ex2 gRNA2 NGS | GTGACTGGAGTTCAGACGTGTGCTCTTCCGATCTCAT | 7 |
| | 2R | CAAACGGGCCCTAAAG | |
| | Ex3 gRNA3 NGSF | ACACTCTTTCCCTACACGACGCTCTTCCGATCTGG | 8 |
| | | CTGGTTTATCAACTGCAAA | |
| | Ex3 gRNA3 NGSR | GTGACTGGAGTTCAGACGTGTGCTCTTCCGATCTAAA | 9 |
| | | AAGAGCTCTACTCTGATGTCAA | |
| | Ex4 gRNA4 NGSF | ACACTCTTTCCCTACACGACGCTCTTCCGATCTCG | 10 |
| | | TCAAAGTTAGACCATTTCCTC | |
| | Ex4 gRNA4 NGSR | GTGACTGGAGTTCAGACGTGTGCTCTTCCGATCTCCT | 11 |
| | | CTGATGACAGTACAAATAACAGG | |
| | Ex5 gRNA5 NGSF | ACACTCTTTCCCTACACGACGCTCTTCCGATCTAG | 12 |
| | | TCGGGGCTTACCCAAATA | |
| | Ex5 gRNA5 NGSR | GTGACTGGAGTTCAGACGTGTGCTCTTCCGATCTGGA | 13 |
| | | AAATCAAATCGGCTGAA | |
| | Ex8 gRNA8 | ACACTCTTTCCCTACACGACGCTCTTCCGATCTAT | 14 |
| | NGS2F | GGT TCGAAGATGGCATTC | |
| | Ex8 gRNA8 | GTGACTGGAGTTCAGACGTGTGCTCTTCCGATCTAAG | 15 |
| | NGS2R | CATCTCCGTCAATCACA | |
| | Ex11 gRNA11 | ACACTCTTTCCCTACACGACGCTCTTCCGATCTTC | 16 |
| | NGSF | CTCTTAAGCACAAATTGAGTTC | |
| | Ex11 gRNA11 | GTGACTGGAGTTCAGACGTGTGCTCTTCCGATCTAAC | 17 |
| | NGSR | AAATGCTCTGGGCATAAA | |
| | Ex13 gRNA13a | ACACTCTTTCCCTACACGACGCTCTTCCGATCTTT | 18 |
| | NGSF | GTGTAATCCCCGGCTAAATA | |
| | Ex13 gRNA13a | GTGACTGGAGTTCAGACGTGTGCTCTTCCGATCTTTC | 19 |
| | NGSR | ATGCTGAAGACAAGGATTC | |
| | Ex13 gRNA13b | ACACTCTTTCCCTACACGACGCTCTTCCGATCTTA | 20 |
| | NGSF | TTTAGCCGGGGATTACACAA | |
| | Ex13 gRNA13b | GTGACTGGAGTTCAGACGTGTGCTCTTCCGATCTCAA | 21 |
| | NGSR | GCCACAAAGCTTTCAAATA | |
| HRM | Exon 3 gRNAF | TATTTGGCAGATGCAGGCCA | 22 |
| | Exon 3 gRNAR | AGAGAGTTCTATAGATCAGCACAAA | 23 |
| | Exon 3 gRNAR | GAACTCTACTCTGATGTCAAC | 24 |
| | Exon 4 gRNAF | CGTCAAAGTTAGACCATTTCCTCTG | 25 |
| | Exon 4 gRNAR | GCCTGTCTCATACCAGTCCC | 26 |
| | Exon 5 gRNAF | ACAGAACCTGTTTGGAGAACT | 27 |
| | Exon 5 gRNAR | GGCATTGCAAAAATCATAGAGTGC | 28 |
| | Exon 13a gRNAF | GATCTCCATTATGAAGCCTAACAGA | 29 |
| | Exon 13a gRNAR | ACCCTTCTACAGGAGACCTTT | 30 |
| | Exon 13b gRNAF | TATTTGGCTTCAATGGAAGGTG | 31 |
| | Exon 13b gRNAR | AAAAGACAGCTTGAAGACAACATTT | 32 |
| Sanger | CsPDSEx1 F | TGCGATATGGTTTCCGAGAT | 33 |
| | CsPDSEx2 R | TCCACAATGCCATACACACC | 34 |
| | CsPDSEx3 F | CTTGTCTCCGACAAATGCAA | 35 |
| | CsPDSEx3 R | GCACAGGTTATTGAGGGTGAA | 36 |
| | CsPDSEx4 F | TTAGACTCCCCTCCCAAGGT | 37 |
| | CsPDSEx4 R | TCCAGGCAAAGTGAAAAAGC | 38 |
| | CsPDSEx5 F | TGTGGAAACTTGTGGGAGTG | 39 |
| | CsPDSEx5 R | TCTAGTTTCTCAGCATGGCCTA | 40 |
| | CsPDSEx8 F | TGGATATGCTTTGCTGGTTG | 41 |
| | CsPDSEx8 R | GCCAAATTGCATTTCAAAGA | 42 |
| | CsPDSEx11 F | GCCCATTGCTTTTAGCATTT | 43 |
| | CsPDSEx11 R | GCCACATGCATTCCATAGGT | 44 |
| | CsPDSEx13 F | CGTCAAAACGCCAAGGTTAG | 45 |
| | CsPDSEx13 R | GCAGCAAGCAGCACATAGTC | 46 |
| PCR | Cas9 F | CTTCAACTGCTTAACGGTAAC | 47 |
| | Cas9 R | GATTTCTACCCATTCTTGAAG | 48 |
| | CsACTIN F | CTCGATATTGGCTTCCCTCA | 49 |
| | CsACTIN R | AGACATGGCCTCCACAAAAC | 50 |
| Taq-Man | GFP F | GCCGAACGGGAAATTGTC | 51 |
| | GFP R | GCAGTCTCAAGTTCTTGCTCATAG | 52 |
| | GFP-Probe | CGTGACATGAAGGAGAAGCTTGCTTA | 53 |
| | CsACTIN F | CGTGACCACCTTCACCTA | 54 |
| | CsACTIN R | GCATGGCGGACTTGAAGAAG | 55 |
| | CsACTIN-Probe | CGTGCAGTGCTTCAGCCGCTAC | 56 |

### Example 2

### The CRISPRUS Genetic System

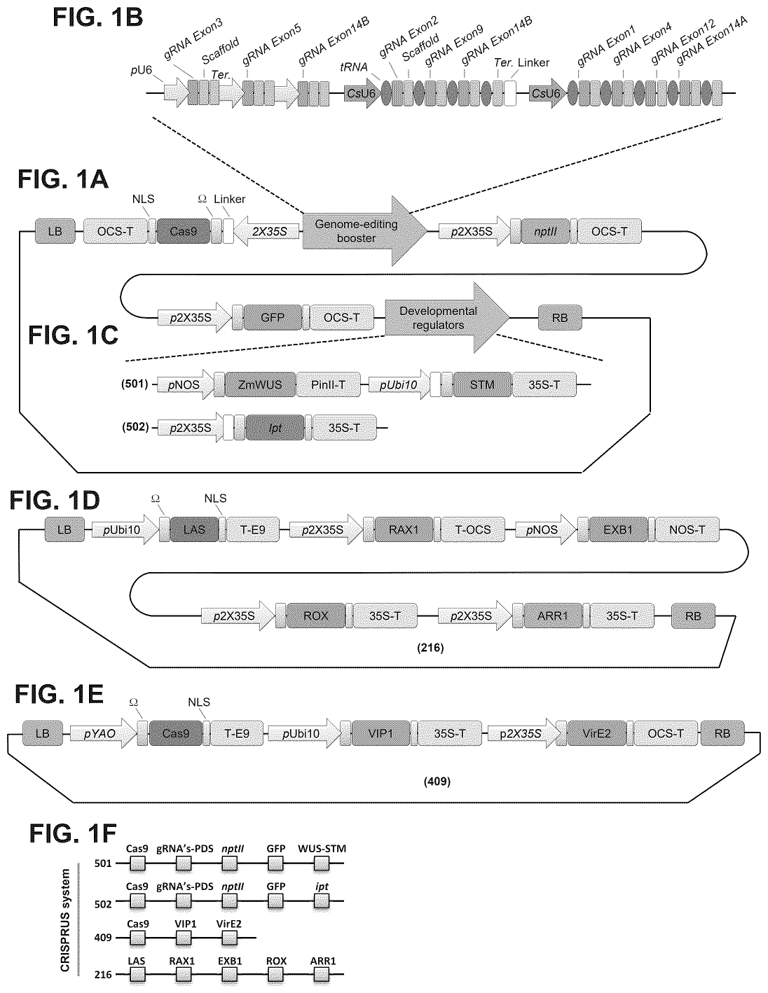

In the pursuit of generating genome-edited Citrus plants in-planta, the CRISPR/Cas9-sgRNA expression system was used and applied to the decapitated site of young seedlings. The primary objective was to induce de novo edited meristem that later would develop to form a complete independent shoot (Maher et al., 2020). The target gene selected for genome editing is the PHYTOENE DESATURASE (CsPDS), known for producing albino phenotype when mutated (Qin et al., 2007), thereby serving as a convenient visual marker for successful gene editing. Nine guide RNAs (sgRNAs) were designed to target the coding region of eight different exons of CsPDS (Cs stands for Citrus sinensis). These gRNAs were expressed under the control of both a short NekU6 promoter (pU6) and a double polycistronic-tRNA-gRNA system (PTG), under the CsU6 endogenous promoter, as described by (Huang et al., 2020), forming a comprehensive genome-editing booster cassette (SEQ ID NO: 57, FIG. 1B).

In general, the CRISPRUS (a combination of CRISPR and Citrus, as it is denoted in a specific embodiment of the invention) system comprises genes specializing in three essential functions: editing, meristem induction, and Agrobacterium delivery, expressed in four vectors (FIGS. 1A-D). The first two vectors include a genome-editing booster cassette and transcriptional unit of Cas9, selection markers, and developmental regulators (FIG. 1A). The regulators used are either WUSCHEL (WUS) together with SHOOT MERISTEMLESS (STM, FIG. 1C, plasmid 501), or a gene encoding isopentyl transferase (ipt, FIG. 1C, plasmid 502). The third vector (plasmid 216) is the shoot induction and regenerated cassette, features the genes: LATERAL SUPPRESSOR (LAS), REGULATOR OF AXILLARY MERISTEMS (RAX1), EXCESSIVE BRANCHES1 (EXB1), REGULATOR OF AXILLARY MERISTEM FORMATION (ROX) and RESPONSE REGULATOR1 (ARR1) (Greb et al., 2003, Keller et al., 2006, Yang et al., 2012, Guo and Qin, 2016, Cao and Jiao, 2020). These genes act upstream of WUS and STM, while EXB1, RAX1, and LAS function specifically in axillary meristem formation ((Cao and Jiao, 2020), FIG. 1D, plasmid 216). The combination of these genes designed to further enhance the de novo meristem acquisition and to promote the overall shoot induction.

The final vector aimed at improving Cas9 expression and Agro delivery (plasmid 409). To increase the spatial, temporal, and overall transient activity of Cas9, the YAO promoter was employed to drive the expression of Cas9 in cells undergoing cell division (FIG. 1E, plasmid 409 (Yan et al., 2015)). It was suggested that a combination of 2x35S and YAO promoters (FIGS. 1C-D, plasmids 501, 502, 409) would provide better coverage for the Cas9 in the transient expression system for improving editing. In addition, the YAO-Cas9 unit was followed by an overexpression VIrE2-INTERACTING PROTEIN (VIP1) and the Agrobacterium effector protein VirE2 to facilitate a more efficient Agrobacterium T-DNA delivery (FIG. 1E, Lacroix and Citovsky, 2013, Gelvin, 2017, Lacroix and Citovsky, 2019). These four vectors (FIG. 1F, plasmids 501, 502, 216, and 409) were each inserted into independent Agrobacterium, and co-injected onto the decapitated site of young seedlings.

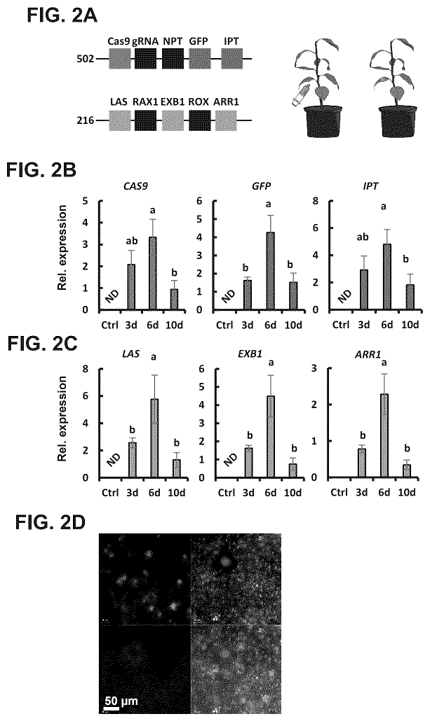

The efficiency of the system depends on generating sufficient expression level and on simultaneous activity of the different Agrobacterium applied. The study was initiated by confirming the in-planta activity of the CRISPRUS system. Agro infiltration of young leaves was performed, and the expression levels of two vectors, 502 and 216, were tracked at three time points (FIG. 2A). The expression levels of three genes within each vector were significantly elevated compared to control (non-treated) leaves (FIGS. 2B-C). The peak expression was observed on day 6 post-infiltration, followed by a reduction on day 10. At the end of the experiment, GFP fluorescence was detected, further validating the functionality of the 502 vector.

To examine the simultaneous activity of independently applied agrobacteria, a fluorescence assay was done using three distinct fluorescent proteins, each delivered by different Agrobacterium. The results demonstrate that following agroinfiltration, all tested cells displayed fluorescence of the three agrobacteria used, as evidenced by merged images of GFP, mCherry and CFP (not shown). Hence, the co-infection delivery by a collection of Agrobacterium demonstrated to be an efficient strategy, facilitating a cooperative expression within specific cells or tissues.

### Example 3

### Regeneration Efficiency

Citrus plant regeneration usually presents a challenging task. To address this, the present inventors initially evaluated the regeneration efficiency in response to treatment with the CRISPRUS system (FIGS. 3A-G). The application of the CRISPRUS system relied on previous studies with adjustments (Zhang et al., 2017b, Rizwan et al., 2021). Soil-grown seedlings of Carrizo citrange were excised approximately 5 cm above the ground (epicotyls). Then, the seedlings were subjected to Agrobacterium using plastic tips and transferred to dark conditions. Ten days later, the seedlings were moved to the light, and regeneration was notable about 20 days later (FIG. 3A). The seedlings were treated either with an empty Agro lacking any DR's, or with the CRISPRUS system (FIGS. 3B-C). Shoot induction was measured in seedlings originating from the side of epicotyls (below the cut site, green color) and compared with those regenerating from the top cut site, believed to be stimulated by the system (FIGS. 3C-D). Under control conditions (Cut, no treatment), 38.1% of the seedlings regenerated from the top (FIG. 3D). However, when subjected to Agro lacking developmental regulators, the regeneration efficiency dropped to 5.6% (FIGS. 3B, 3D). In contrast, treatment with CRISPRUS system significantly enhanced regeneration efficiency, attaining rates as high as 75% (FIGS. 3C-D). Furthermore, among the seedlings regenerating from the top and treated using CRISPRUS system, 62.5% displayed even higher shoot induction, with at least two shoots per plant (FIG. 3D). In comparison, control seedlings (no agro, and Agro with no DR's) displayed considerably lower rates, 8.6% and 2.5%, respectively (FIG. 3D). The CRISPRUS system demonstrated the potential to promote more than ten shoots per plant (FIG. 3F), with 20 being the highest recorded shoot count in the experiments. However, in some cases, excessive regeneration occasionally led to over shooting and callus formation (FIG. 3G).

### Example 4

### Evaluation of Genome Editing Efficiency

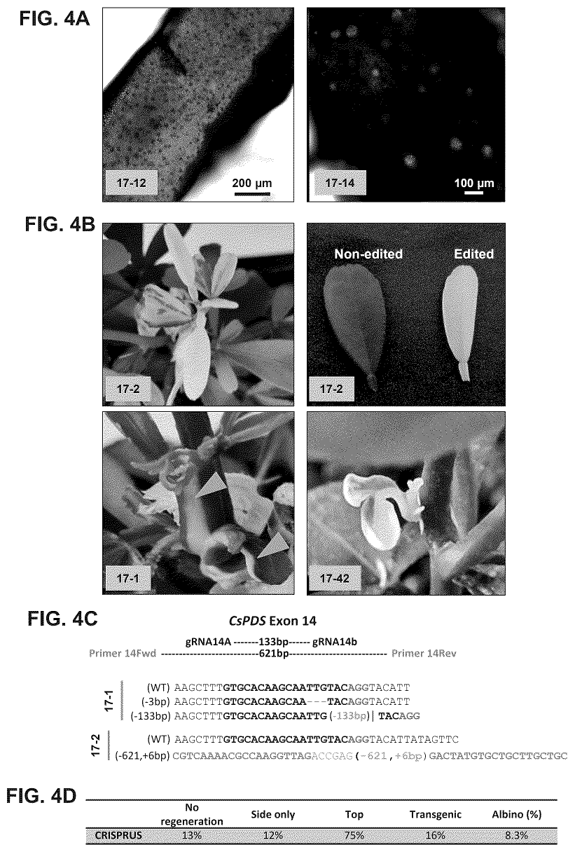

Next, the genome editing potential of the CRISPRUS system was evaluated on Carrizo plants (FIGS. 4A-D) while using guides RNA's (sgRNA's) targeting the carotenoid biosynthetic gene PHYTOENE DESATURASE (CsPDS, FIG. 1B). This approach resulted in a spectrum of outcomes, including transgenic shoots and transgenic with genome-editing (FIGS. 4A-D). Out of all the plants tested, the present inventors were able to identify plants that are either fully—(line 17-12) or partly (line 17-14) transgenic, as indicated by GFP fluorescence (FIG. 4A). Regarding genome editing, out of 60 plants, three chimeric plants were identified that were partly albino (FIG. 4B, lines 17-1, 17-2 and 17-42), displaying small and large deletions (FIG. 4C). Interestingly, it is noted that one chimeric plant contained a white axil adjacent to a green (WT) axil extending from base to the tip of the shoot (FIG. 4B, 17-1), suggesting that editing can occur at very early stages during shoot development, most likely in progenitor cells. Of all plants tested, 16% were transgenic and 8.3% albino. Among the mutated (albino) plants, #17-2 was found to be transgene-free, even though a selection step was applied. However, all edited plants observed were chimeric to some degree.

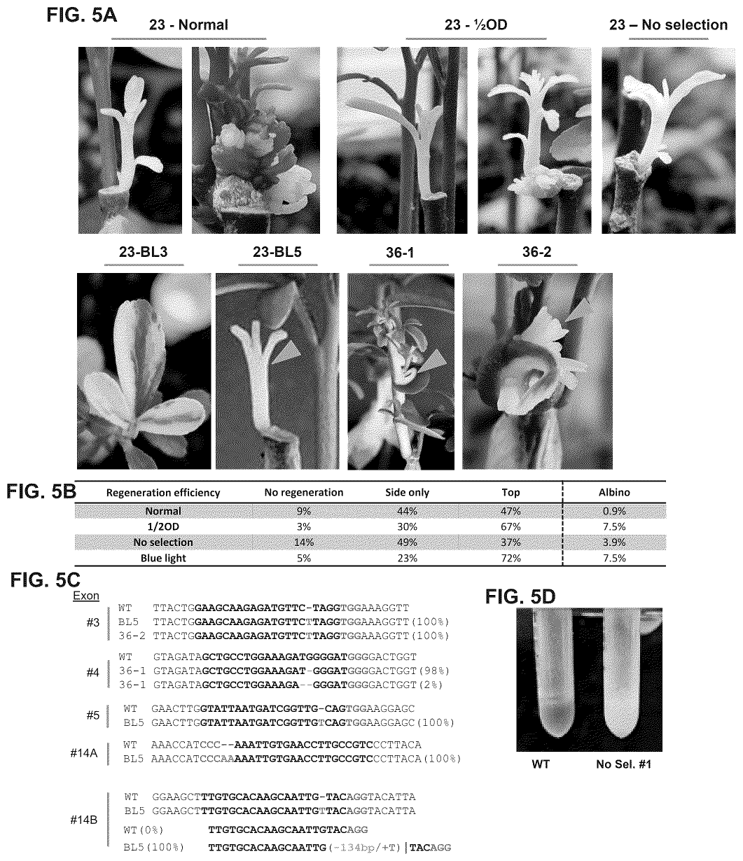

To increase the likelihood of obtaining a complete edited plant, the present inventors used 400 plants and subjected them to four treatments. Treatments were as follows: standard OD (as in FIGS. 4A-D), blue light pre-exposure for improved regeneration as recently shown (Dong et al., 2023), treatment without selection (the transgene-free passive approach), to search for non-transgenic edited shoots, and ½OD (optical density) to reduce the level of over regeneration and callus formation that was sometimes observed (FIG. 3G). All treatments have produced albino plants (FIGS. 5A, 5D). While some were chimeric, others were fully mutated, displaying a complete base-to-tip albino phenotype, confirming the method's ability to generate fully mutated edited shoots. The plants in ½OD and blue light treatments showed the highest regeneration rates, as well as higher rates of albino plants (FIG. 5B). Notably; albino shoots were produced even when the selection step was not applied (transgene-free passive approach, 3.9%, FIGS. 5A, and 5B). Deep sequencing of a complete albino (23-BL5) and the white segment of the sectorial chimeric plants (36-1, 36-2) obtained from additional experiments further verified the editing efficiency (FIG. 5C). For example, in 36-1 and 36-2 display a deletion and insertion, respectively (FIG. 5C, Exons #3, and #4). In the case of shoot 23-BL5, a bi-allelic mutation was detected in exon #14B (FIG. 5C), characterized by deletion and insertion, and, more importantly, lacks any traces of wild-type sequence. Moreover, this sample was impaired in multi-alleles, as indicated in the sequences of exons #3, #5, #14A, and #14B (FIG. 5B). Taken together, the CRISPRUS system produces fully developed mutated shoots that can later develop to form a new variety.

### Example 5

### Cross-Species Application in Citrus

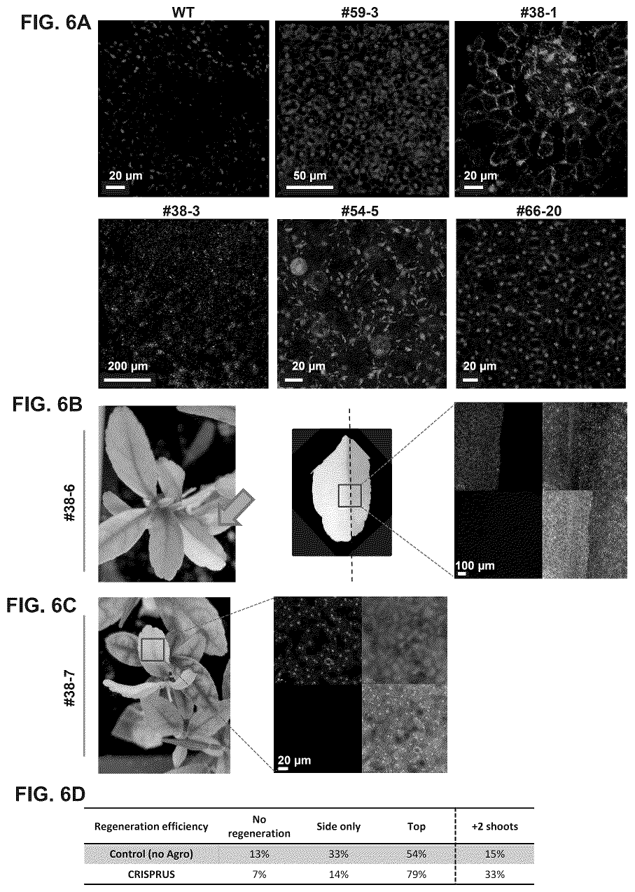

The next step was to assess the Citrus cross-species applicability of our system. In addition to Carrizo, the Duncan grapefruit cultivar (Citrus paradisi Macf.) was subjected to treatment. Similar to the findings in Carizzo, transgenic plants were identified, as evidenced by GFP fluorescence (FIG. 6A). These plants were completely transgenic, further indicating the CRISPRUS system's ability to target initial meristematic cells. In contrast to Carrizo, genome editing of CsPDS presented a difficult challenge when applied to Duncan. Duncan's regenerated shoots demonstrated high sensitivity to the impaired CsPDS gene, often leading to the abortion of the mutated shoots. Despite these challenges, mutated (chimeric) shoots were identified when detected at the early stages of shoot emergence (FIGS. 6B-C). For example, leaves that displayed chimera with the presence of white and green areas, divided by the primary mid-vein, displayed GFP fluorescence in the white side of the leaf, while the green side, holding a functional PDS gene, remained non-transgenic (FIG. 6B, #38-6). In cases where the chimera degree was less pronounced, a milky-yellowish coloration was observed. It was confirmed by confocal imaging, revealing a mixture of GFP-expressing- and WT cells (FIG. 6B, line #38-7). This indicates a partial transgenic (and edited) state, with both edited and non-edited cells coexisting within the same leaf.

### Example 6

### The Active Approach to Produce Transgene-Free Genome-Edited Plants

A primary goal while using the method of some embodiments of the invention is to generate transgene-free elite cultivars. It is evidenced that while using the in-planta CRISPRUS system, transgene-free gene editing can be achieved via the passive approach of skipping the selection step. However, the efficiency of this approach is still restrictive. In efforts to increase the efficiency of eliminating T-DNA integration, the present inventors aimed for a direct (active) approach to prevent integration. It is suggested that targeting a specific polymerase that is required to complete the integration step would facilitate thus. Following a DNA break, the 5′ end of the TDNA aligns with the plant's exposed DNA strand. To complete the integration, a complementary strand is synthesizes by DNA polymerase θ (Polθ, Genestier et al., 1994, Gelvin, 2021). Previously, it was shown that two independent Arabidopsis mutants impaired in Polθ display rare integration events (Van Kregten et al., 2016). Equipped with this knowledge, it was suggested that silencing of the endogenous expression of CsPolθ in parallel to the application of the CRISPRUS system would reduce integration rates.

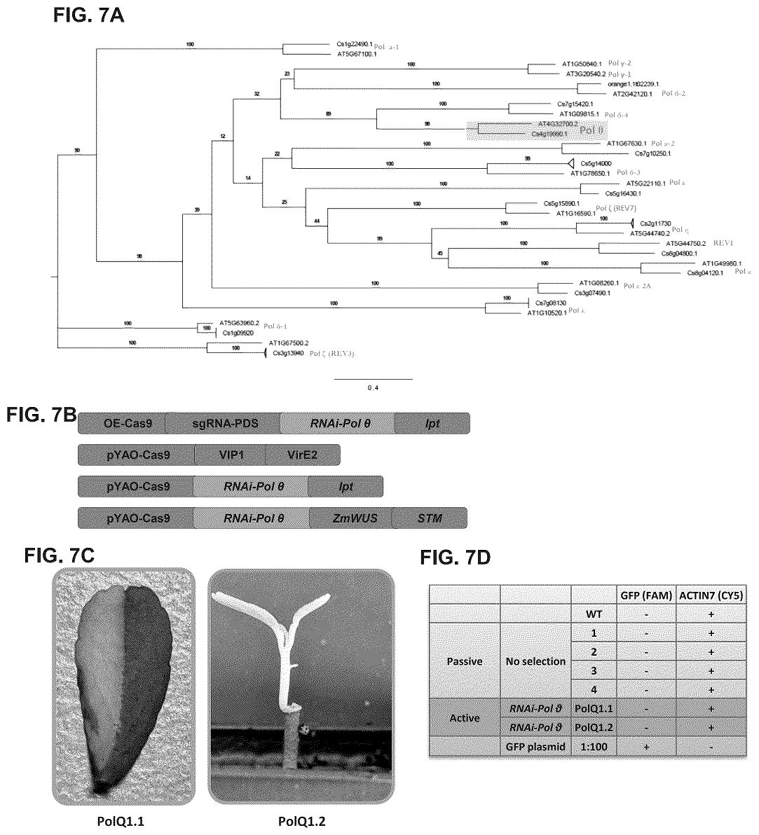

First, the AtPolθ homolog in Citrus was sought. To do so, a phylogenetic tree of DNA polymerases in Citrus was produced, compared with the known DNA polymerases in Arabidopsis (FIG. 7A, Pedroza-Garcia et al., 2019)). The analysis revealed that the homolog of AtPolθ (AT4G32700) in Citrus is Cs4g199990 (98%, FIG. 7A, named CsPolθ). A plasmid for a short-term silencing of the CsPolθ was developed, by using RNAi and added to the plasmids used in the system (FIG. 7B, orange). Indeed, by silencing the CsPolθ, albino plants impaired in the CsPDS gene were obtained that are transgene-free. A summarized data of TaqMan results for transgene presence is provided (FIG. 7D).

### Example 7

### Vacuum-Based Introduction

In addition to the decapitated seedling method which was generally used an exemplified herein, a vacuum-assisted approach was explored to assess its potential for facilitating gene editing. To this end, the floral dip transformation protocol, originally developed for Arabidopsis ovaries, was adapted and optimized for application in Citrus. In eight-month-old Carrizo plants, the apical and all axillary meristems were removed, after which the plants were immersed in an Agrobacterium suspension carrying the CRISPR/Cas system, followed by vacuum infiltration (FIGS. 8A-B). Three weeks post-treatment, new shoots emerged (FIG. 8A), including an albino shoot containing a CSPDS mutation. This shoot exhibited GFP fluorescence (FIG. 8B) and carried a large genomic deletion spanning exon 2 to exon 13 of the CSPDS gene (˜18 kb). These findings demonstrate that the described genome editing system is compatible with multiple delivery approaches for generating gene-edited Citrus shoots.

### Example 8

### Transgene-Free Genome-Editing

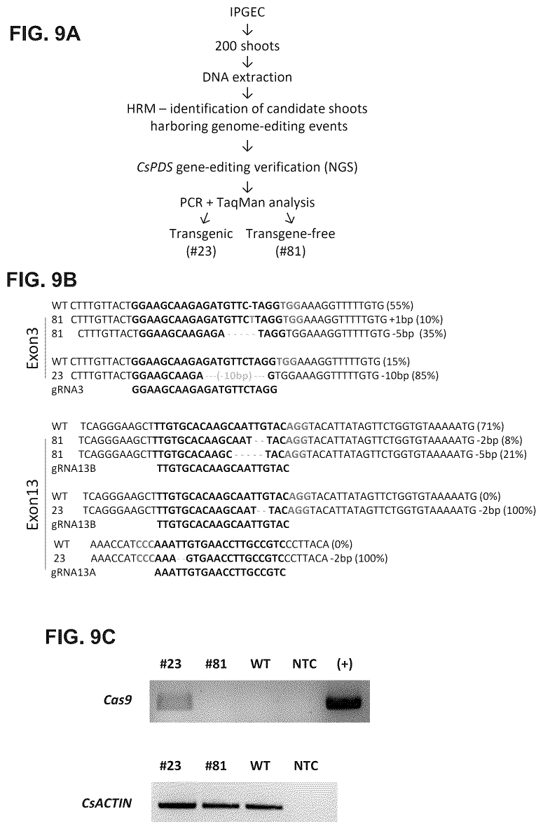

The ultimate goal for an editing system in fruit trees is a system that gives rise to edited, yet non-transgenic shoots. The present inventors speculated that the enhanced T-DNA delivery in the IPGEC system may generate transient expression levels that are sufficient to lead to genome editing, without integration of T-DNA, resulting in transgene-free genome-edited shoots. To address this possibility, a population of 200 Carrizo shoots was generated following IPGEC treatment, and the resulting shoots were screened for editing mutants using the sensitive high-resolution melting (HRM) method (FIG. 9A). The present inventors were able to identify chimeric CsPDS-impaired shoots, which were further verified by next generation sequencing (NGS). Among the chimeric edited lines, the present inventors detected sample #81 that was found to harbor editing-based deletions in exon13 (−2 bp, −5 bp) in 30% of the sequenced reads and editing-based deletions/insertions in exon3 (+1 bp, −5 bp) in 45% of the sequences (FIG. 9B). A PCR reaction and the sensitive TaqMan analyses were used to verify that these shoots were transgene-free (FIG. 9C). Thus, the present inventors demonstrate that transgene-free editing is achievable.

### Example 9

### Cultivar-To-Developmental Regulator Interaction Determine Regeneration and Transgenic Efficiencies

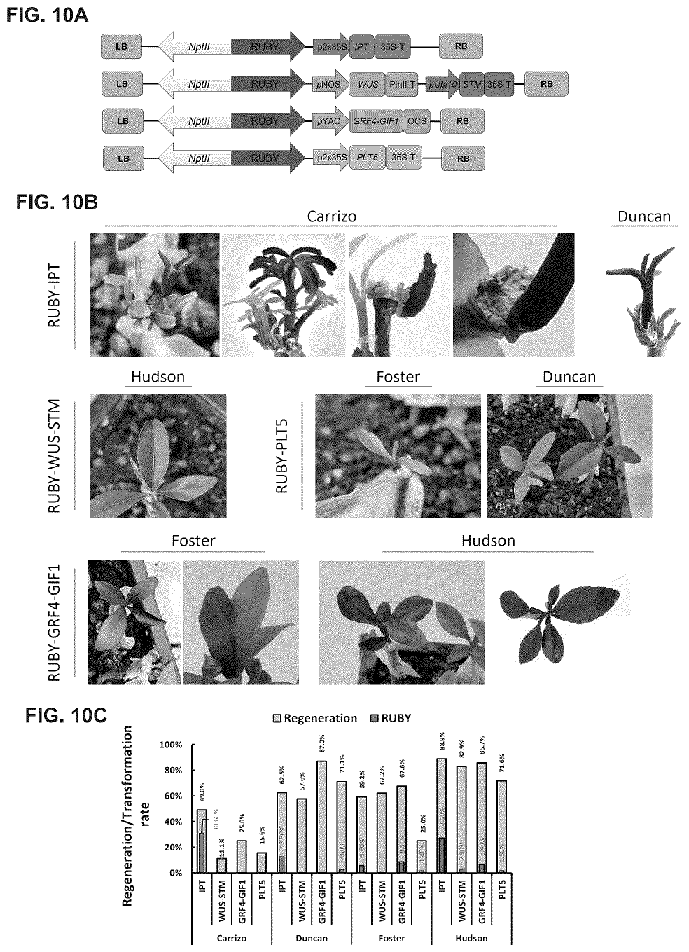

As demonstrated above, the use of developmental regulators significantly enhances regeneration rates and genome-edited shoot production. The present inventors therefore tested the potential impact of additional developmental factors on shoot emergence efficiency. PLT5 and GRF4-GIF1. Hence, the present inventors studied the effect of these factors, alongside IPT and WUS-STM DRs used in the IPGEC system, on the regeneration and transformation efficiency of four different Citrus cultivars (Carrizo, Duncan, Foster, and Hudson). For convenient screening of the positively transformed shoots, a cassette expressing the betalain biosynthetic genes (RUBY) was used that lead to accumulation of the pink/purple metabolite betalain (He et al., 2020; Kumar et al., 2022; Polturak et al., 2016). Four independent vectors that contain the RUBY cassette together with either IPT, WUS-STM, GRF4-GIF1, or PLT5 transcriptional units, driven by different promoters, were assembled (FIG. 10A), and expressed in-planta using the IPGEC protocol. Regeneration and transformation rates were analyzed 60 days following infection with Agrobacterium (FIG. 10B-C). Carrizo generally displayed relatively low regeneration rates compared with the other cultivars, in all DRs tested (FIG. 10C). IPT was found to be the most efficient DR in Carrizo and Hudson, while in Duncan and Foster, it was the GRF4-GIF1, with rates above 90% (FIG. 10C). In contrast, using the PLT5 in Carrizo and Foster displayed limited efficiency where regeneration rates were relatively low (15.6% and 25%, respectively).

Concerning transformation rates, variable results were observed throughout this survey. Generally, the highest number of shoots displaying betalain accumulation was observed when IPT served as a DR. This was the case for Carrizo, Duncan, and Hudson (FIG. 10C). In Foster, however, RUBY-expressing shoots were most prominent when GRF4-GIF1 was used. Overall, these results demonstrate the capability of the In-planta methodology to yield transgenic plants in a variety of species and emphasizes the need to match the best performing DR for every species. Thus, a prior optimization step assessing the efficiency of the different DRs may (but not mandatory to the procedure) enhance the rate of success of IPGEC towards the end-game of producing transgene-free edited plants (FIG. 9A-C).

Strategies to obtain transgene-free genome-edited plants continue to evolve. In annual crops, this process is relatively straightforward whereby following validation of editing, transgenes can be segregated-out through self-pollination or backcrossing. However, this method is impractical for perennial trees such as Citrus due to their high heterozygosity, the lengthy juvenile phase, and, in many elite varieties, impaired or aborted sexual reproduction resulting in seedless fruit. Recent innovations have sought to address these challenges. One approach involves protoplast editing by the Cas9-sgRNA ribonucleoprotein system, followed by regeneration (Lin et al., 2018; Su et al., 2023). Another potentially effective strategy combines the co-editing of a selectable gene (the acetolactate synthase gene for herbicide resistance) together with the gene of interest (Huang et al., 2023; Jia et al., 2024). Our study provides an indication that transgene-free editing can potentially be achieved via transient expression of DRs and genome editing components in-planta, thus saving both time and effort as well as devoid of the risks associated with somaclonal variation (Mei et al., 2024). In our current work, we demonstrated the ability to produce chimeric plants that are both transgene-free and edited. For example, sample #81 shoot exhibits multiallelic editing, with 45% and 30% editing observed in exons3 and 13, respectively, while genetic screens verified it to be transgene-free (FIG. 9C). We note that shoot chimerism is not a limiting factor in producing fully edited plants, as the conversion of chimeric radiation-mutagenesis-derived mutants to fully mutated ones is a common practice in Citrus breeding programs achieved by successive bud grafting. (Goldenberg et al., 2014; Vardi et al., 2008). Furthermore, high throughput screening techniques facilitating much larger editing experiments are likely to yield a variety of edited events, including uniformly edited shoots. Therefore, although the generation of transgene-free, genome-edited Citrus plants requires additional effort to ensure methodological reproducibility, it is a feasible approach that paves the way to develop commercially viable non-GMO edited cultivars.

Thus, the ability to produce chimeric plants that are both transgene-free and edited was exemplified. For example, sample #81 shoot exhibits multiallelic editing, with 45% and 30% editing observed in exons3 and 13, respectively, while genetic screens verified it to be transgene-free (FIG. 9C). It will be appreciated that shoot chimerism is not a limiting factor in producing fully edited plants, as the conversion of chimeric radiation-mutagenesis-derived mutants to fully mutated ones is a common practice in Citrus breeding programs achieved by successive bud grafting. (Goldenberg et al., 2014; Vardi et al., 2008).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Application

Alvarez, D., Cerda-Bennasser, P., Stowe, E., Ramirez-Torres, F., Capell, T., Dhingra, A. and Christou, P. (2021) Fruit crops in the era of genome editing: closing the regulatory gap. Plant Cell Rep, 40, 915-930.
Campa, M., Miranda, S., Licciardello, C., Lashbrooke, J. G., Dalla Costa, L., Guan, Q., Spök, A. and Malnoy, M. (2023) Application of new breeding techniques in fruit trees. Plant Physiology, kiad374.
Cao, X. and Jiao, Y. (2020) Control of cell fate during axillary meristem initiation. Cellular and Molecular Life Sciences, 77, 2343-2354.
Cody, J. P., Maher, M. F., Nasti, R. A., Starker, C. G., Chamness, J. C. and Voytas, D. F. (2023) Direct delivery and fast-treated Agrobacterium co-culture (Fast-TrACC) plant transformation methods for Nicotiana benthamiana. Nature protocols, 18, 81-107.
Dong, H., Wang, J., Song, X., Hu, C., Zhu, C., Sun, T., Zhou, Z., Hu, Z., Xia, X. and Zhou, J. (2023) HY5 functions as a systemic signal by integrating BRC1-dependent hormone signaling in tomato bud outgrowth. Proceedings of the National Academy of Sciences, 120, e2301879120.
Dutt, M., Mahmoud, L. M., Nehela, Y., Grosser, J. W. and Killiny, N. (2022) The Citrus sinensis TILLER ANGLE CONTROL 1 (CsTAC1) gene regulates tree architecture in sweet oranges by modulating the endogenous hormone content. Plant Sci, 323, 111401.
Eshed Williams, L. (2021) Genetics of shoot meristem and shoot regeneration. Annual Review of Genetics, 55, 661-681.
Gelvin, S. B. (2017) Integration of Agrobacterium T-DNA into the plant genome. Annual review of genetics, 51, 195-217.
Gelvin, S. B. (2021) Plant DNA Repair and Agrobacterium T-DNA Integration. Int J Mol Sci, 22.
Genestier, L., Dearden-Badet, M. T., Bonnefoy-Berard, N., Lizard, G. and Revillard, J. P. (1994) Cyclosporin A and FK506 inhibit activation-induced cell death in the murine WEHI-231 B cell line. Cell Immunol, 155, 283-291.
Greb, T., Clarenz, O., Schäfer, E., Müller, D., Herrero, R., Schmitz, G. and Theres, K. (2003) Molecular analysis of the LATERAL SUPPRESSOR gene in Arabidopsis reveals a conserved control mechanism for axillary meristem formation. Gene Dev, 17, 1175-1187.
Guo, D. and Qin, G. (2016) EXB1/WRKY71 transcription factor regulates both shoot branching and responses to abiotic stresses. Plant signaling & behavior, 11, e1150404.
Hamada, H., Linghu, Q., Nagira, Y., Miki, R., Taoka, N. and Imai, R. (2017) An in planta biolistic method for stable wheat transformation. Sci Rep, 7, 11443.
Hao, S., Zhang, Y., Li, R., Qu, P. and Cheng, C. (2024) Agrobacterium-mediated in planta transformation of horticultural plants: Current status and future prospects. Scientia Horticulturae, 325, 112693.
Huang, X., Wang, Y. and Wang, N. (2022a) Base Editors for Citrus Gene Editing. Front Genome Ed, 4, 852867.
Huang, X., Wang, Y. and Wang, N. (2022b) Highly efficient generation of canker-resistant sweet orange enabled by an improved CRISPR/Cas9 system. Front Plant Sci, 12, 769907.
Huang, X., Wang, Y., Xu, J. and Wang, N. (2020) Development of multiplex genome editing toolkits for Citrus with high efficacy in biallelic and homozygous mutations. Plant molecular biology, 104, 297-307.
Jia, H., Xu, J., Orbović, V., Zhang, Y. and Wang, N. (2017) Editing Citrus genome via SaCas9/sgRNA system. Front Plant Sci, 8, 2135.
Keller, T., Abbott, J., Moritz, T. and Doerner, P. (2006) Arabidopsis REGULATOR OF AXILLARY MERISTEMS1 controls a leaf axil stem cell niche and modulates vegetative development. The Plant Cell, 18, 598-611.
Lacroix, B. and Citovsky, V. (2013) Characterization of VIP1 activity as a transcriptional regulator in vitro and in planta. Scientific Reports, 3, 1-7.
Lacroix, B. and Citovsky, V. (2019) Pathways of DNA transfer to plants from Agrobacterium tumefaciens and related bacterial species. Annual review of phytopathology, 57, 231-251.
Lian, Z., Nguyen, C. D., Liu, L., Wang, G., Chen, J., Wang, S., Yi, G., Wilson, S., Ozias-Akins, P. and Gong, H. (2022) Application of Developmental Regulators to Improve In-Planta or In Vitro Transformation in Plants. Plant Biotechnology Journal.
Maher, M. F., Nasti, R. A., Vollbrecht, M., Starker, C. G., Clark, M. D. and Voytas, D. F. (2020) Plant gene editing through de novo induction of meristems. Nature biotechnology, 38, 84-89.
Mahmoud, L. M., Kaur, P., Stanton, D., Grosser, J. W. and Dutt, M. (2022) A cationic lipid mediated CRISPR/Cas9 technique for the production of stable genome edited Citrus plants. Plant methods, 18, 33.
Nasti, R. A. and Voytas, D. F. (2021) Attaining the promise of plant gene editing at scale. Proceedings of the National Academy of Sciences, 118, e2004846117.
Nerva, L., Dalla Costa, L., Ciacciulli, A., Sabbadini, S., Pavese, V., Dondini, L., Vendramin, E., Caboni, E., Perrone, I. and Moglia, A. (2023) The role of Italy in the use of advanced plant genomic techniques on fruit trees: state of the art and future perspectives. Int J Mol Sci, 24, 977.
Parajuli, S., Huo, H., Gmitter Jr, F. G., Duan, Y., Luo, F. and Deng, Z. (2022) Editing the CsDMR6 gene in Citrus results in resistance to the bacterial disease Citrus canker. Horticulture Research, 9, uhac082.
Pedroza-Garcia, J.-A., De Veylder, L. and Raynaud, C. (2019) Plant DNA polymerases. Int J Mol Sci, 20, 4814.
Qin, G., Gu, H., Ma, L., Peng, Y., Deng, X. W., Chen, Z. and Qu, L.-J. (2007) Disruption of phytoene desaturase gene results in albino and dwarf phenotypes in Arabidopsis by impairing chlorophyll, carotenoid, and gibberellin biosynthesis. Cell research, 17, 471-482.
Rizwan, H. M., Yang, Q., Yousef, A. F., Zhang, X., Sharif, Y., Kaijie, J., Shi, M., Li, H., Munir, N. and Yang, X. (2021) Establishment of a novel and efficient Agrobacterium-mediated in planta transformation system for passion fruit (Passiflora edulis). Plants, 10, 2459.
Sarrion-Perdigones, A., Vazquez-Vilar, M., Palací, J., Castelijns, B., Forment, J., Ziarsolo, P., Blanca, J., Granell, A. and Orzaez, D. (2013) GoldenBraid 2.0: a comprehensive DNA assembly framework for plant synthetic biology. Plant physiology, 162, 1618-1631.
Song, G.-q., Prieto, H. and Orbovic, V. (2019) Agrobacterium-mediated transformation of tree fruit crops: methods, progress, and challenges. Front Plant Sci, 10, 226.
Van Kregten, M., de Pater, S., Romeijn, R., van Schendel, R., Hooykaas, P. J. and Tijsterman, M. (2016) T-DNA integration in plants results from polymerase-θ-mediated DNA repair. Nature plants, 2, 1-6.
Yan, L., Wei, S., Wu, Y., Hu, R., Li, H., Yang, W. and Xie, Q. (2015) High-Efficiency Genome Editing in Arabidopsis Using YAO Promoter-Driven CRISPR/Cas9 System. Mol Plant, 8, 1820-1823.
Yang, F., Wang, Q., Schmitz, G., Müller, D. and Theres, K. (2012) The bHLH protein ROX acts in concert with RAX1 and LAS to modulate axillary meristem formation in Arabidopsis. The Plant Journal, 71, 61-70.
Zhang, T.-Q., Lian, H., Zhou, C.-M., Xu, L., Jiao, Y. and Wang, J.-W. (2017a) A two-step model for de novo activation of WUSCHEL during plant shoot regeneration. The Plant Cell, 29, 1073-1087.
Zhang, Y.-y., Zhang, D.-m., Zhong, Y., Chang, X.-j., Hu, M.-l. and Cheng, C.-z. (2017b) A simple and efficient in planta transformation method for pommelo (Citrus maxima) using Agrobacterium tumefaciens. Scientia Horticulturae, 214, 174-179.
Zhou, J., Li, D., Wang, G., Wang, F., Kunjal, M., Joldersma, D. and Liu, Z. (2020) Application and future perspective of CRISPR/Cas9 genome editing in fruit crops. Journal of integrative plant biology, 62, 269-286.

What is claimed is:

1. A method of producing a genomically edited plant or part thereof comprising contacting the plant or part thereof with the nucleic acid construct or construct system under conditions which allow editing and regeneration, wherein the plant is non-transgenic at T₀, wherein the nucleic acid construct or construct system comprises:
(i) at least one nucleic acid sequence encoding a genome editing agent for introducing a sequence variation in a target gene of interest in a plant cell; and
(ii) at least one nucleic acid sequence encoding at least one gene being a developmental regulator.

2. The method of claim 1, wherein progeny of the genomically edited plant is non-transgenic.

3. The method of claim 1, further comprising (iii) at least one nucleic acid sequence encoding at least one gene for facilitating Agrobacterium delivery into nuclei of the plant cells.

4. The method of claim 3, wherein (iii) is selected from the group consisting of VIP1 and VirE2.

5. The method of claim 1, further comprising (iv) at least one nucleic acid sequence for preventing T-DNA integration.

6. The method of claim 5, wherein said at least one nucleic acid sequence for preventing T-DNA integration is directed against Polymerase θ (Polθ).

7. The method of claim 5, wherein said at least one nucleic acid sequence for preventing T-DNA integration is an RNA silencing agent or a genome editing agent.

8. The method of claim 1, wherein the genome editing agent is of the CRISPR-Cas system.

9. The method of claim 1, wherein said at least one developmental regulator is selected from the group consisting of GRF4, IPT, GIF1, WUS, STM and PLT5.

10. The method of claim 1, wherein said nucleic acid construct or construct system comprises cis acting regulatory elements operatively linked to at least one nucleic acid sequences.

11. The method of claim 10, wherein said cis acting regulatory elements which are operably linked to (i) are active in cells undergoing cell division (e.g., Yao).

12. The method of claim 1, wherein said nucleic acid construct or construct system comprises a selection gene.

13. The method of claim 1, wherein the plant is a tree.

14. The method of claim 1, wherein the plant is a Citrus.

15. The method of claim 1, wherein said conditions comprise exposing to blue light prior to said contacting.

16. The method of claim 1, wherein said conditions comprise maintaining said plant or part thereof in the dark to promote regeneration following said contacting and prior to exposing to light.

17. The method of claim 1, wherein said contacting is at an apical site of a seedling.

18. The method of claim 1, wherein said contacting is by in planta immersing or vacuum.

19. The method of claim 1, further comprising grafting the plant part.

20. A plant or part thereof obtainable according to the method of claim 1.


SEQUENCE LISTING (97 sequences)

SEQ ID NO: 1 - protein, 7 aa, synthetic construct
PKKKRKV

SEQ ID NO: 2 - DNA, 20 bp, synthetic construct
  source 1..20: mol_type = other DNA
tgcgatatggtttccgagat

SEQ ID NO: 3 - DNA, 20 bp, synthetic construct
  source 1..20: mol_type = other DNA
gcagcaagcagcacatagtc

SEQ ID NO: 4 - DNA, 53 bp, synthetic construct
  source 1..53: mol_type = other DNA
acactctttccctacacgacgctcttccgatctccgagatagtgaaccgatgg

SEQ ID NO: 5 - DNA, 55 bp, synthetic construct
  source 1..55: mol_type = other DNA
gtgactggagttcagacgtgtgctcttccgatctggtagtccacacaaaccacct

SEQ ID NO: 6 - DNA, 55 bp, synthetic construct
  source 1..55: mol_type = other DNA
acactctttccctacacgacgctcttccgatctggaagctgcttacttgtcttcg

SEQ ID NO: 7 - DNA, 53 bp, synthetic construct
  source 1..53: mol_type = other DNA
gtgactggagttcagacgtgtgctcttccgatctcatcaaacgggccctaaag

SEQ ID NO: 8 - DNA, 54 bp, synthetic construct
  source 1..54: mol_type = other DNA
acactctttccctacacgacgctcttccgatctggctggtttatcaactgcaaa

SEQ ID NO: 9 - DNA, 59 bp, synthetic construct
  source 1..59: mol_type = other DNA
gtgactggagttcagacgtgtgctcttccgatctaaaaagagctctactctgatgtcaa

SEQ ID NO: 10 - DNA, 56 bp, synthetic construct
  source 1..56: mol_type = other DNA
acactctttccctacacgacgctcttccgatctcgtcaaagttagaccatttcctc

SEQ ID NO: 11 - DNA, 60 bp, synthetic construct
  source 1..60: mol_type = other DNA
gtgactggagttcagacgtgtgctcttccgatctcctctgatgacagtacaaataacagg

SEQ ID NO: 12 - DNA, 53 bp, synthetic construct
  source 1..53: mol_type = other DNA
acactctttccctacacgacgctcttccgatctagtcggggcttacccaaata

SEQ ID NO: 13 - DNA, 54 bp, synthetic construct
  source 1..54: mol_type = other DNA
gtgactggagttcagacgtgtgctcttccgatctggaaaatcaaatcggctgaa

SEQ ID NO: 14 - DNA, 53 bp, synthetic construct
  source 1..53: mol_type = other DNA
acactctttccctacacgacgctcttccgatctatggttcgaagatggcattc

SEQ ID NO: 15 - DNA, 54 bp, synthetic construct
  source 1..54: mol_type = other DNA
gtgactggagttcagacgtgtgctcttccgatctaagcatctccgtcaatcaca

SEQ ID NO: 16 - DNA, 57 bp, synthetic construct
  source 1..57: mol_type = other DNA
acactctttccctacacgacgctcttccgatcttcctcttaagcacaaattgagttc

SEQ ID NO: 17 - DNA, 55 bp, synthetic construct
  source 1..55: mol_type = other DNA
gtgactggagttcagacgtgtgctcttccgatctaacaaatgctctgggcataaa

SEQ ID NO: 18 - DNA, 55 bp, synthetic construct
  source 1..55: mol_type = other DNA
acactctttccctacacgacgctcttccgatctttgtgtaatccccggctaaata

SEQ ID NO: 19 - DNA, 56 bp, synthetic construct
  source 1..56: mol_type = other DNA
gtgactggagttcagacgtgtgctcttccgatctttcatgctgaagacaaggattc

SEQ ID NO: 20 - DNA, 55 bp, synthetic construct
  source 1..55: mol_type = other DNA
acactctttccctacacgacgctcttccgatcttatttagccggggattacacaa

SEQ ID NO: 21 - DNA, 56 bp, synthetic construct
  source 1..56: mol_type = other DNA
gtgactggagttcagacgtgtgctcttccgatctcaagccacaaagctttcaaata

SEQ ID NO: 22 - DNA, 20 bp, synthetic construct
  source 1..20: mol_type = other DNA
tatttggcagatgcaggcca

SEQ ID NO: 23 - DNA, 25 bp, synthetic construct
  source 1..25: mol_type = other DNA
agagagttctatagatcagcacaaa

SEQ ID NO: 24 - DNA, 21 bp, synthetic construct
  source 1..21: mol_type = other DNA
gaactctactctgatgtcaac

SEQ ID NO: 25 - DNA, 25 bp, synthetic construct
  source 1..25: mol_type = other DNA
cgtcaaagttagaccatttcctctg

SEQ ID NO: 26 - DNA, 20 bp, synthetic construct
  source 1..20: mol_type = other DNA
gcctgtctcataccagtccc

SEQ ID NO: 27 - DNA, 21 bp, synthetic construct
  source 1..21: mol_type = other DNA
acagaacctgtttggagaact

SEQ ID NO: 28 - DNA, 24 bp, synthetic construct
  source 1..24: mol_type = other DNA
ggcattgcaaaaatcatagagtgc

SEQ ID NO: 29 - DNA, 25 bp, synthetic construct
  source 1..25: mol_type = other DNA
gatctccattatgaagcctaacaga

SEQ ID NO: 30 - DNA, 21 bp, synthetic construct
  source 1..21: mol_type = other DNA
acccttctacaggagaccttt

SEQ ID NO: 31 - DNA, 22 bp, synthetic construct
  source 1..22: mol_type = other DNA
tatttggcttcaatggaaggtg

SEQ ID NO: 32 - DNA, 25 bp, synthetic construct
  source 1..25: mol_type = other DNA
aaaagacagcttgaagacaacattt

SEQ ID NO: 33 - DNA, 20 bp, synthetic construct
  source 1..20: mol_type = other DNA
tgcgatatggtttccgagat

SEQ ID NO: 34 - DNA, 20 bp, synthetic construct
  source 1..20: mol_type = other DNA
tccacaatgccatacacacc

SEQ ID NO: 35 - DNA, 20 bp, synthetic construct
  source 1..20: mol_type = other DNA
cttgtctccgacaaatgcaa

SEQ ID NO: 36 - DNA, 21 bp, synthetic construct
  source 1..21: mol_type = other DNA
gcacaggttattgagggtgaa

SEQ ID NO: 37 - DNA, 20 bp, synthetic construct
  source 1..20: mol_type = other DNA
ttagactcccctcccaaggt

SEQ ID NO: 38 - DNA, 20 bp, synthetic construct
  source 1..20: mol_type = other DNA
tccaggcaaagtgaaaaagc

SEQ ID NO: 39 - DNA, 20 bp, synthetic construct
  source 1..20: mol_type = other DNA
tgtggaaacttgtgggagtg

SEQ ID NO: 40 - DNA, 22 bp, synthetic construct
  source 1..22: mol_type = other DNA
tctagtttctcagcatggccta

SEQ ID NO: 41 - DNA, 20 bp, synthetic construct
  source 1..20: mol_type = other DNA
tggatatgctttgctggttg

SEQ ID NO: 42 - DNA, 20 bp, synthetic construct
  source 1..20: mol_type = other DNA
gccaaattgcatttcaaaga

SEQ ID NO: 43 - DNA, 20 bp, synthetic construct
  source 1..20: mol_type = other DNA
gcccattgcttttagcattt

SEQ ID NO: 44 - DNA, 20 bp, synthetic construct
  source 1..20: mol_type = other DNA
gccacatgcattccataggt

SEQ ID NO: 45 - DNA, 20 bp, synthetic construct
  source 1..20: mol_type = other DNA
cgtcaaaacgccaaggttag

SEQ ID NO: 46 - DNA, 20 bp, synthetic construct
  source 1..20: mol_type = other DNA
gcagcaagcagcacatagtc

SEQ ID NO: 47 - DNA, 21 bp, synthetic construct
  source 1..21: mol_type = other DNA
cttcaactgcttaacggtaac

SEQ ID NO: 48 - DNA, 21 bp, synthetic construct
  source 1..21: mol_type = other DNA
gatttctacccattcttgaag

SEQ ID NO: 49 - DNA, 20 bp, synthetic construct
  source 1..20: mol_type = other DNA
ctcgatattggcttccctca

SEQ ID NO: 50 - DNA, 20 bp, synthetic construct
  source 1..20: mol_type = other DNA
agacatggcctccacaaaac

SEQ ID NO: 51 - DNA, 18 bp, synthetic construct
  source 1..18: mol_type = other DNA
gccgaacgggaaattgtc

SEQ ID NO: 52 - DNA, 24 bp, synthetic construct
  source 1..24: mol_type = other DNA
gcagtctcaagttcttgctcatag

SEQ ID NO: 53 - DNA, 26 bp, synthetic construct
  source 1..26: mol_type = other DNA
cgtgacatgaaggagaagcttgctta

SEQ ID NO: 54 - DNA, 18 bp, synthetic construct
  source 1..18: mol_type = other DNA
cgtgaccaccttcaccta

SEQ ID NO: 55 - DNA, 20 bp, synthetic construct
  source 1..20: mol_type = other DNA
gcatggcggacttgaagaag

SEQ ID NO: 56 - DNA, 22 bp, synthetic construct
  source 1..22: mol_type = other DNA
cgtgcagtgcttcagccgctac

SEQ ID NO: 57 - DNA, 3323 bp, synthetic construct
  source 1..3323: mol_type = other DNA
gtcaggagcctgcaggtagatcgctcctcgacggagtgatcaaaagtcccacatcgatcaggtgatatatagcagcttagtttatataatgatagagtcgacatagcgattgaagcaagagatgttctagggttttagagctagaaatagcaagttaaaataaggctagtccgttatcaacttgaaaaagtggcaccgagtcggtgctttttttcgatgagctcggatccctaggagtgatcaaaagtcccacatcgatcaggtgatatatagcagcttagtttatataatgatagagtcgacatagcgattgtattaatgaccggttgcaggttttagagctagaaatagcaagttaaaataaggctagtccgttatcaacttgaaaaagtggcaccgagtcggtgctttttttcgatgagctcggatccctaggagtgatcaaaagtcccacatcgatcaggtgatatatagcagcttagtttatataatgatagagtcgacatagcgattttgtgcacaagcaattgtacgttttagagctagaaatagcaagttaaaataaggctagtccgttatcaacttgaaaaagtggcaccgagtcggtgctttttttcgctgtcaggagcgctcaggagccggttgaatttgattgttgtttgatgtttaggtatgcttacaattttttactaaataaggtaataattgttccttttattttattcatttagcttggtacttgtcgtaatctatctccgtcaatgtcagcttcttctgtgtcggaagaagctgaaaaatgttgttgaaggccctttcatttattattatcattattatgtttaattttgttcggcaaatgtgtactaatttggttcgaatcgttcgatctcataatacaaacatgtaatgagttatttatgacgtcatggatgcggttagtttgcgtggtctgaaatttcaaccaaattattcaatcatggtggtggccggctggtgcctatcccttgattgaaatatgcaattaaaattctcgtaataatgttgatttgttctatatacttgaattgatggtattaattattgttataaaagcactaacttgtttgagaaaggggataactaaaaggtaataataataataataaaataaagaaaaggcccaacacatgggcgccctatctgttgggccaaatgccataagaggatccagctagcccgttgaagaaaatcccacatcgaaagaaaaaactgaataaacatggtctatatatacaaggactcccaggttggttgaacaaagcaccagtggtctagtggtagaatagtaccctgccacggtacagacccgggttcgattcccggctggtgcaaaagttgtaattgctggtgcgtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagaacacattcagtcactggggtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcattgtgcacaagcaattgtacgtttaagagctatgctggaaacagcatagcaagtttaaataaggctagtccgttatcaacttgaaaaagtggcaccgagtcggtgctttttttttcctgcaggtagatcgctcctcgacaagcttcgctgtcaggagcgctcaggagccggttgaatttgattgttgtttgatgtttaggtatgcttacaattttttactaaataaggtaataattgttccttttattttattcatttagcttggtacttgtcgtaatctatctccgtcaatgtcagcttcttctgtgtcggaagaagctgaaaaatgttgttgaaggccctttcatttattattatcattattatgtttaattttgttcggcaaatgtgtactaatttggttcgaatcgttcgatctcataatacaaacatgtaatgagttatttatgacgtcatggatgcggttagtttgcgtggtctgaaatttcaaccaaattattcaatcatggtggtggccggctggtgcctatcccttgattgaaatatgcaattaaaattctcgtaataatgttgatttgttctatatacttgaattgatggtattaattattgttataaaagcactaacttgtttgagaaaggggataactaaaaggtaataataataataataaaataaagaaaaggcccaacacatgggcgccctatctgttgggccaaatgccataagaggatccagctagcccgttgaagaaaatcccacatcgaaagaaaaaactgaataaacatggtctatatatacaaggactcccaggttggttgaacaaagcaccagtggtctagtggtagaatagtaccctgccacggtacagacccgggttcgattcccggctggtgcagttaaaacgaggacaaggaagtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagctgcctggaaagatggggagtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagtcggcatacacacttagaagtttaagagctatgctggaaacagcatagcaagtttaaataaggctagtccgttatcaacttgaaaaagtggcaccgagtcggtgcaacaaagcaccagtggtctagtggtagaatagtaccctgccacggtacagacccgggttcgattcccggctggtgcagacggcaaggttcacaatttgtttaagagctatgctggaaacagcatagcaagtttaaataaggctagtccgttatcaacttgaaaaagtggcaccgagtcggtgctttttttttcctgcaggtagatcgctcctcgac

SEQ ID NO: 58 - DNA, 34 bp, synthetic construct
  source 1..34: mol_type = other DNA
aagctttgtgcacaagcaattgtacaggtacatt

SEQ ID NO: 59 - DNA, 31 bp, synthetic construct
  source 1..31: mol_type = other DNA
aagctttgtgcacaagcaatacaggtacatt

SEQ ID NO: 60 - DNA, 28 bp, synthetic construct
  source 1..28: mol_type = other DNA
aagctttgtgcacaagcaattgtacagg

SEQ ID NO: 61 - DNA, 41 bp, synthetic construct
  source 1..41: mol_type = other DNA
aagctttgtgcacaagcaattgtacaggtacattatagttc

SEQ ID NO: 62 - DNA, 46 bp, synthetic construct
  source 1..46: mol_type = other DNA
cgtcaaaacgccaaggttagaccgaggactatgtgctgcttgctgc

SEQ ID NO: 63 - DNA, 36 bp, synthetic construct
  source 1..36: mol_type = other DNA
ttactggaagcaagagatgttctaggtggaaaggtt

SEQ ID NO: 64 - DNA, 37 bp, synthetic construct
  source 1..37: mol_type = other DNA
ttactggaagcaagagatgttcttaggtggaaaggtt

SEQ ID NO: 65 - DNA, 37 bp, synthetic construct; identical to SEQ ID NO: 64
  source 1..37: mol_type = other DNA

SEQ ID NO: 66 - DNA, 38 bp, synthetic construct
  source 1..38: mol_type = other DNA
gtagatagctgcctggaaagatggggatggggactggt

SEQ ID NO: 67 - DNA, 37 bp, synthetic construct
  source 1..37: mol_type = other DNA
gtagatagctgcctggaaagatgggatggggactggt

SEQ ID NO: 68 - DNA, 36 bp, synthetic construct
  source 1..36: mol_type = other DNA
gtagatagctgcctggaaagagggatggggactggt

SEQ ID NO: 69 - DNA, 37 bp, synthetic construct
  source 1..37: mol_type = other DNA
gaacttggtattaatgatcggttgcagtggaaggagc

SEQ ID NO: 70 - DNA, 38 bp, synthetic construct
  source 1..38: mol_type = other DNA
gaacttggtattaatgatcggttgtcagtggaaggagc

SEQ ID NO: 71 - DNA, 37 bp, synthetic construct
  source 1..37: mol_type = other DNA
aaaccatcccaaattgtgaaccttgccgtcccttaca

SEQ ID NO: 72 - DNA, 39 bp, synthetic construct
  source 1..39: mol_type = other DNA
aaaccatcccaaaaattgtgaaccttgccgtcccttaca

SEQ ID NO: 73 - DNA, 37 bp, synthetic construct
  source 1..37: mol_type = other DNA
ggaagctttgtgcacaagcaattgtacaggtacatta

SEQ ID NO: 74 - DNA, 38 bp, synthetic construct
  source 1..38: mol_type = other DNA
ggaagctttgtgcacaagcaattgttacaggtacatta

SEQ ID NO: 75 - DNA, 23 bp, synthetic construct
  source 1..23: mol_type = other DNA
ttgtgcacaagcaattgtacagg

SEQ ID NO: 76 - DNA, 23 bp, synthetic construct
  source 1..23: mol_type = other DNA
ttgtgcacaagcaattgtacagg

SEQ ID NO: 77 - DNA, 47 bp, synthetic construct
  source 1..47: mol_type = other DNA
ctttgttactggaagcaagagatgttctaggtggaaaggtttttgtg

SEQ ID NO: 78 - DNA, 48 bp, synthetic construct
  source 1..48: mol_type = other DNA
ctttgttactggaagcaagagatgttcttaggtggaaaggtttttgtg

SEQ ID NO: 79 - DNA, 42 bp, synthetic construct
  source 1..42: mol_type = other DNA
ctttgttactggaagcaagagataggtggaaaggtttttgtg

SEQ ID NO: 80 - DNA, 47 bp, synthetic construct; identical to SEQ ID NO: 77
  source 1..47: mol_type = other DNA

SEQ ID NO: 81 - DNA, 37 bp, synthetic construct
  source 1..37: mol_type = other DNA
ctttgttactggaagcaagagtggaaaggtttttgtg

SEQ ID NO: 82 - DNA, 21 bp, synthetic construct
  source 1..21: mol_type = other DNA
ggaagcaagagatgttctagg

SEQ ID NO: 83 - DNA, 60 bp, synthetic construct
  source 1..60: mol_type = other DNA
tcagggaagctttgtgcacaagcaattgtacaggtacattatagttctggtgtaaaaatg

SEQ ID NO: 84 - DNA, 58 bp, synthetic construct
  source 1..58: mol_type = other DNA
tcagggaagctttgtgcacaagcaattacaggtacattatagttctggtgtaaaaatg

SEQ ID NO: 85 - DNA, 55 bp, synthetic construct
  source 1..55: mol_type = other DNA
tcagggaagctttgtgcacaagctacaggtacattatagttctggtgtaaaaatg

SEQ ID NO: 86 - DNA, 20 bp, synthetic construct
  source 1..20: mol_type = other DNA
ttgtgcacaagcaattgtac

SEQ ID NO: 87 - DNA, 60 bp, synthetic construct; identical to SEQ ID NO: 83
  source 1..60: mol_type = other DNA

SEQ ID NO: 88 - DNA, 58 bp, synthetic construct; identical to SEQ ID NO: 84
  source 1..58: mol_type = other DNA

SEQ ID NO: 89 - DNA, 20 bp, synthetic construct
  source 1..20: mol_type = other DNA
ttgtgcacaagcaattgtac

SEQ ID NO: 90 - DNA, 37 bp, synthetic construct; identical to SEQ ID NO: 71
  source 1..37: mol_type = other DNA

SEQ ID NO: 91 - DNA, 35 bp, synthetic construct
  source 1..35: mol_type = other DNA
aaaccatcccaaagtgaaccttgccgtcccttaca

SEQ ID NO: 92 - DNA, 20 bp, synthetic construct
  source 1..20: mol_type = other DNA
aaattgtgaaccttgccgtc

SEQ ID NO: 93 - DNA, 92 bp, synthetic construct
  source 1..92: mol_type = other DNA
tccttcacctctttctacgtggcctaacaactatggagtttgggttgatgagtttgaagacataggacttgtagactgtttggacaaaactt

SEQ ID NO: 94 - DNA, 93 bp, synthetic construct
  source 1..93: mol_type = other DNA
tccttcacctctttctacgtggcctaacaactatggagtttgggttgatgagtttgaagatcataggacttgtagactgtttggacaaaactt

SEQ ID NO: 95 - DNA, 93 bp, synthetic construct
  source 1..93: mol_type = other DNA
tccttcacctctttctacgtggcctaacaactatggagtttgggttgatgagtttgaagaacataggacttgtagactgtttggacaaaactt

SEQ ID NO: 96 - DNA, 92 bp, synthetic construct; identical to SEQ ID NO: 93
  source 1..92: mol_type = other DNA

SEQ ID NO: 97 - DNA, 15 bp, synthetic construct
  source 1..15: mol_type = other DNA
gtttggacaaaactt